(19) United States
(12) Patent Application Publication
(10) Pub. No.: US 2021/0059837 A1
(43) Pub. Date: Mar. 4, 2021

(54) PATIENT SPECIFIC GUIDE FOR ACETABULAR REAMER AND IMPACTOR

(71) Applicant: DePuy Synthes Products, Inc., Raynham, MA (US)

(72) Inventor: James M. Rhodes, Warsaw, IN (US)

(21) Appl. No.: 16/559,756

(22) Filed: Sep. 4, 2019

(51) Int. Cl. A61F 2/46; A61B 17/16; A61B 17/17; A61F 2/30; A61B 34/10

(52) U.S. Cl. CPC A61F 2/4609; A61B 17/1666; A61B 2017/568; A61F 2/30942; A61B 34/10; A61B 17/1746

(57) ABSTRACT

A customized patient-specific orthopaedic instrument for facilitating implantation of an acetabular cup prosthesis in a coxal bone of a patient and method of use is disclosed. The customized patient-specific orthopaedic instrument includes a customized patient-specific acetabular guide. The customized patient-specific acetabular guide includes a longitudinal passageway for an acetabular reamer and/or an impactor and a plurality of arms with attached mounting pads. Each pad of the guide is positioned relative to the body based on the contours of the coxal bone of the patient and a predetermined degree of anteversion and inclination angles of the acetabular cup prosthesis when implanted in the patient's coxal bone.

### TECHNICAL FIELD

The present disclosure relates generally to customized patient-specific orthopaedic surgical instruments and, more particularly, to customized patient-specific acetabular orthopaedic surgical instruments.

### BACKGROUND

Joint arthroplasty is a well-known surgical procedure by which a diseased and/or damaged natural joint is replaced by a prosthetic joint. For example, in a hip arthroplasty surgical procedure, a prosthetic hip replaces a patient's natural hip. A typical prosthetic hip includes an acetabular orthopaedic prosthesis and/or femoral head orthopaedic prosthesis. A typical acetabular orthopaedic prosthesis includes an acetabular cup, which is secured to the patient's natural acetabulum, and an associated polymer bearing or ring.

To facilitate the replacement of the natural joint with an acetabular orthopaedic prosthesis, orthopaedic surgeons may use a variety of orthopaedic surgical instruments such as, for example, reamers, guide members, drills, and/or other surgical instruments. Typically, such orthopaedic surgical instruments are generic with respect to the patient such that the same orthopaedic surgical instrument may be used on a number of different patients during similar orthopaedic surgical procedures.

### SUMMARY

According to one aspect, a customized patient-specific orthopaedic instrument for facilitating implantation of an acetabular cup prosthesis in a coxal bone of a patient may include a customized patient-specific acetabular guide assembly. The customized patient-specific acetabular guide assembly may include a guide member having a longitudinal passageway defined therethrough. The customized patient-specific acetabular reaming guide may also include a plurality of arms extending from the guide member. Additionally, the customized patient-specific acetabular guide assembly may include a plurality of mounting pads configured to contact a coxal bone of a patient. Each mounting pad of the plurality of mounting pads may be coupled to a corresponding arm of the plurality of arms. Additionally, each mounting pad of the plurality of mounting pads may be positioned relative to the guide member based on a predetermined degree of version and inclination angles of the acetabular cup prosthesis when implanted in the patient's coxal bone and on the contour of the coxal bone of the patient. Each mounting pad can be patient-specific so as to fit over a unique portion of the patient's coxal bone, and can be fabricated using any suitable additive manufacturing technique.

In some embodiments, each mounting pad of the plurality of mounting pads may include a bottom, bone-facing surface having a customized patient-specific positive contour configured to receive a portion of the patient's coxal bone having a corresponding negative contour. Additionally, in some embodiments, the longitudinal passageway of the guide member may be sized to receive a tool shaft that can be selectively coupled to a reamer and an impactor. Alternatively, the longitudinal passageway of the guide member can receive a bone guide pin.

In some embodiments, the guide member may include a bottom, bone-facing surface and each mounting pad of the plurality of mounting pads may include a top surface. The bottom, bone-facing surface of the guide member may be coplanar or non-coplanar with respect to a plane defined by the top surface of least one of the plurality of mounting pads. In some embodiments, the plurality of mounting pads includes a first mounting pad having a first top surface defining a first plane and a second mounting pad having a second top surface defining a second plane. In such embodiments, the bottom, bone-facing surface of the guide member, the first top surface, and the second top surface may be parallel and non-coplanar with respect to each other. Additionally, in some embodiments, each mounting pad of the plurality of mounting pads may include a bottom surface. The bottom, bone-facing surface of the guide member may be positioned medially with respect to the bottom surface of each mounting pad of the plurality of mounting pads when the customized patient-specific acetabular reaming guide is positioned in contact with the patient's coxal bone.

Additionally, in some embodiments, each mounting pad of the plurality of mounting pads may have a longitudinal length substantially different from each other. The guide member may also include a sidewall and each arm of the plurality of arms may include a bottom surface. Each bottom surface of the plurality of arms may define an angle with respect to the sidewall of the guide member that is different in magnitude with respect to the angle defined by each other bottom surface of the plurality of arms. Additionally, in some embodiments, an angle may be defined between each arm of the plurality of arms with respect to another adjacent arm of the plurality of arms when viewed in the top plan view. Each of such angles may be different in magnitude from each other. Additionally, each mounting pad of the plurality of mounting pads may be spaced apart from the guide member, when viewed in the top plan view, a distance different in magnitude with respect to the distance defined by each other mounting pad of the plurality of mounting pads.

In some embodiments, each arm of the plurality of arms may be coupled to the guide member via a joint such that each arm is movable relative to the guide member. Additionally or alternatively, each mounting pad of the plurality of mounting pads may be coupled to the corresponding arm via a joint such that each mounting pad is movable relative to the corresponding arm. In some embodiments, the plurality of arms may comprise at least three arms extending from the guide member. Additionally, in some embodiments, each mounting pad of the plurality of mounting pads may include a longitudinal passageway defined therein, each of the longitudinal passageways of the plurality of mounting pads being sized to receive a bone guide pin.

According to another aspect, a customized patient-specific orthopaedic instrument for facilitating implantation of an acetabular cup prosthesis in a coxal bone of a patient may include a customized patient-specific acetabular reaming guide. The customized patient-specific acetabular guide assembly may include a guide member having a longitudinal passageway defined therethrough, a plurality of arms coupled to the guide member via corresponding joints such that each arm of the plurality of arms is separately movable with respect to the guide member and a plurality of mounting pads configured to contact a coxal bone of a patient. Each mounting pad of the plurality of mounting pads may be coupled to a corresponding arm of the plurality of arms via a corresponding joint such that each mounting pad of the plurality of mounting pads is separately movable with respect to the guide member. Additionally each mounting pad of the plurality of mounting pads may include a bottom, bone-facing surface having a customized patient-specific negative contour configured to receive a portion of the patient's coxal bone having a corresponding positive contour. In some embodiments, each mounting pad of the plurality of mounting pads may include a longitudinal passageway defined therein, each of the longitudinal passageways of the plurality of mounting pads being sized to receive a bone guide pin.

According to a further aspect, a method for performing an orthopaedic bone reaming procedure on a patient's acetabulum to facilitate implantation of an acetabular cup prosthesis in a coxal bone of the patient may include positioning a customized patient-specific acetabular guide assembly on the patient's coxal bone. The customized patient-specific acetabular reaming guide may include a guide member having a longitudinal passageway defined therethrough and a plurality of mounting pads coupled to the guide member and configured to contact the coxal bone of the patient. Each mounting pad of the plurality of mounting pads may be positioned relative to the guide member based on a predetermined degree of version and inclination angles of the acetabular cup prosthesis when implanted in the patient's coxal bone.

The method may include the step of attaching a reamer to the tool shaft, and rotating a tool shaft in the passageway to ream the patient's acetabulum. The method may also include the step of removing the reamer from the tool shaft, attaching an impactor to the tool shaft, and driving the impactor against the acetabular prosthesis to secure the prosthesis to the reamed acetabulum.

In another example, the method may include drilling a pilot hole into the patient's acetabulum using the longitudinal passageway of the guide member as a drill guide. Additionally, the method may include inserting a bone guide pin into the pilot hole formed in the patient's acetabulum. The method may further include advancing a cannulated acetabular reamer over the guide pin. The method may also include reaming the patient's acetabulum with the cannulated acetabular reamer using the bone guide pin as a guide for the cannulated reamer.

According to yet a further aspect, a method for performing an orthopaedic bone reaming procedure on a patient's acetabulum to facilitate implantation of an acetabular cup prosthesis in a coxal bone of the patient may include positioning a customized patient-specific acetabular reaming guide on the patient's coxal bone. The customized patient-specific acetabular reaming guide may include a guide member having a longitudinal passageway defined therethrough and a plurality of mounting pads configured to contact the coxal bone of the patient. Each mounting pad of the plurality of mounting pads may be coupled to the guide member and may have a longitudinal passageway defined therethrough. Each mounting pad of the plurality of mounting pads may be positioned relative to the guide member based on a predetermined degree of version and inclination angles of the acetabular cup prosthesis when implanted in the patient's coxal bone.

The method may include drilling a plurality of pilot holes into the patient's coxal bone using the longitudinal passageways of the plurality of mounting pads as drill guides. The method may also include inserting a bone guide pin through each longitudinal passageway of the plurality of mounting pads and into each of the corresponding pilot holes formed in the patient's coxal bone. Additionally, the method may include securing an acetabular reamer within the longitudinal passageway of the guide member. The method may further include reaming the patient's acetabulum with the acetabular reamer using the plurality of guide pins as guides for the acetabular reamer.

In one embodiment, an acetabular guide assembly can include a generic guide member that includes a guide body and a passageway that extends through the guide body along a central axis. The acetabular guide assembly further includes at least one additively manufactured mounting pad defining a top surface and a bottom surface opposite the top surface, wherein the bottom surface has a patient-specific positive contour that matches a negative contour surface of a coxal bone proximate to an acetabulum. The acetabular guide assembly further includes a plurality of arms that are configured to extend from the guide body to the at least one mounting pad, so as to support the guide member relative to the at least one mounting pad at a predetermined position and orientation. For instance, the central axis of the guide member can have a predetermined relationship with respect to planes of anteversion and inclination. The at least one mounting pad includes a plurality of coupling members that are each configured to couple to at least one of the plurality of arms, and an entirety of the mounting pad is seamless.

In one example, the generic guide member is not patient-specific, and is designed to be used in conjunction with a plurality of mounting pads each having different patient-specific contours.

The at least one mounting pad comprises a plurality of mounting pads whose bottom surface, respectively, is contoured so to fit onto a unique portion of the coxal bone.

The coupling members can have a predetermined spatial relationship with each other such that the central axis of the guide member supported by the mounting pads has the predetermined relationship with respect to the planes of anteversion and inclination.

The upper surfaces of the mounting pads can be substantially coplanar with each other when coupled to the arms, respectively, that in turn are coupled to the guide member.

In one embodiment, the at least one mounting pad includes a plurality of mounting pads. Each of the mounting pads can be uniquely keyed to a corresponding one of the arms of the first acetabular guide assembly so as to be located at a first predetermined location and oriented in a first predetermined orientation. In one example, the mounting pads and arms define respective keyed surfaces, such that each of the mounting pads is configured to be coupled to a respective one of the arms and no other arm. The keyed surfaces allow each of mounting pads to couple to the respective one of the arms in a predetermined orientation, and prevent each of the mounting pads from coupling to the respective one of the arms in any orientation other than the predetermined orientation.

The at least one mounting pad can be configured as a single monolithic mounting pad that includes the plurality of coupling members and at least one patient-specific contour at its bottom surface.

In another embodiment, an acetabular implantation system includes the acetabular guide assembly and at least one of a reamer and an impactor that are configured to selectively couple to a tool shaft that is sized to be received in the passageway, and configured to rotate and translate in the passageway. In one example, the tool shaft has a stop member that is configured to abut the guide member so as to limit translation of the tool shaft in the passageway.

In another embodiment, first and second acetabular guide assemblies each includes a guide body that defines a longitudinal passageway, wherein the guide body of the first acetabular guide assembly is substantially identical to the guide body of the second acetabular guide assembly. The first and second acetabular guide assemblies each further includes a plurality of additively manufactured mounting pads each having respective patient-specific positive contours that match corresponding negative contoured surfaces at unique locations of a coxal bone proximate to an acetabulum. The patient-specific positive contours of the mounting pads of the first acetabular guide assembly are all different than the patient-specific positive contours of the mounting pads of the second acetabular guide assembly. The first and second acetabular guide assemblies each further includes a plurality of arms configured to extend from the guide body to the plurality of mounting pads, wherein the arms of the first acetabular guide assembly are configured to support the guide body of the first acetabular guide assembly at a first predetermined location and orientation with respect to the acetabulum of a first patient, and the arms of the second acetabular guide assembly are configured to support the guide body of the second acetabular guide assembly at a second predetermined location and orientation with respect to the acetabulum of a second patient.

Each of the mounting pads of the first acetabular guide assembly is uniquely keyed to a corresponding one of the arms of the first acetabular guide assembly so as to be located at a first predetermined location and oriented in a first predetermined orientation, each of the mounting pads of the second acetabular guide assembly is uniquely keyed to a corresponding one of the arms of the second acetabular guide assembly so as to be located at a second predetermined location and oriented in a second predetermined orientation. At least one of the second predetermined location and second predetermined orientation is different than the first predetermined location and first predetermined orientation, respectively.

The first and second acetabular guide assemblies can each further include a tool shaft configured to rotate and translate in a passageway of the guide body, the tool shaft further configured to selectively couple to a reamer and an impactor. The tool shaft includes a stop member configured to abut the guide body so as to limit translation of the tool shaft in the passageway.

In another embodiment, a method prepares an acetabulum for an implantation of an acetabular prosthesis. The method includes the step of fitting a bottom surface of at least one additively manufactured mounting pad onto a preplanned portion of a coxal bone proximate to the acetabulum, such that a guide member is supported relative to the at least one mounting pad at a predetermined location and orientation with respective to planes of anteversion and inclination. The method further includes the step of inserting a tool shaft through the passageway. The method further includes the step of rotating the tool shaft about the central axis so as to ream the acetabulum with a reamer attached to the tool shaft.

The method can further include the step of guiding the tool shaft to rotate about the central axis during the rotating step.

The method can further include the steps of removing the reamer from the tool shaft, and attaching an impactor to the tool shaft such that the impactor is translatable along the central axis so as to drive the acetabular prostheses into the acetabulum.

The method can further include the step of fitting multiple contours of at least one additively manufactured mounting pad onto respective unique predetermined locations of the coxal bone.

The method can further include the step of coupling each of the plurality of mounting pads to the respective one of a plurality of arms in a predetermined orientation while preventing each of the plurality of mounting pads from being coupled to the respective one of the plurality of arms in any orientation other than the predetermined orientation. The step of coupling each of the plurality of mounting pads to the respective one of the plurality of arms can be performed prior to the fitting step.

### BRIEF DESCRIPTION OF THE DRAWINGS

The detailed description particularly refers to the following figures, in which:

FIG. 1 is a simplified flow diagram of a method for designing and fabricating a customized patient-specific acetabular orthopaedic surgical instrument;

FIG. 2 is an exploded perspective view of an acetabular guide assembly configured to be coupled to a coxal bone;

FIG. 3 is a perspective view of the acetabular guide assembly illustrated in FIG. 2, shown coupled to the coxal bone;

FIG. 4 is a perspective view of a plurality of mounting pads of the acetabular guide assembly illustrated in FIG. 2;

FIG. 5A is a perspective view of the mounting pads illustrated in FIG. 4 shown coupled to the coxal bone;

FIG. 5B is a side elevation view shown an arm of the acetabular guide assembly aligned to be coupled to one of the mounting pads illustrated in FIG. 5A;

FIG. 6A is a perspective view of other embodiments of mounting pads shown coupled to the coxal bone as illustrated in FIG. 5A;

FIG. 6B is a side elevation view of another example of an acetabular guide assembly illustrated in FIG. 1, but including movable arms;

FIG. 7A is a top plan view of the acetabular guide assembly illustrated in FIG. 1, show including arms that are equidistantly spaced from each other;

FIG. 7B is a top plan view showing the arms of the acetabular guide assembly variably spaced from each other;

FIG. 8 is a side elevation view of the acetabular guide assembly of FIG. 1;

FIG. 9A is a top perspective view of another embodiment of a mounting pad;

FIG. 9B is a bottom perspective view of the mounting pad illustrated in FIG. 9A;

FIG. 9C is a bottom perspective view of the mounting pad illustrated in FIGS. 9A-9B, shown coupled to a coxal bone;

FIG. 10 is a simplified flow diagram of a method for performing an acetabular orthopaedic surgical procedure in one example;

FIG. 11 is a side elevation view of a reamer for use in the method of claim 10;

FIG. 12 is a side elevation view of an acetabular prosthesis positioned for implantation in one example;

FIG. 13 is a simplified flow diagram of another embodiment of a method for performing an acetabular orthopaedic surgical procedure;

FIG. 14 is a side elevation view of a cannulated reamer for use in the method of FIG. 13; and

FIG. 15 is a side elevation view of an acetabular prosthesis positioned for implantation using a guide pin secured to the patient's bone via use of the customized acetabular guide assembly illustrated in FIG. 2.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims. Further, the term “at least one” stated structure as used herein can refer to a single one of the stated structure and more than one of the stated structure.

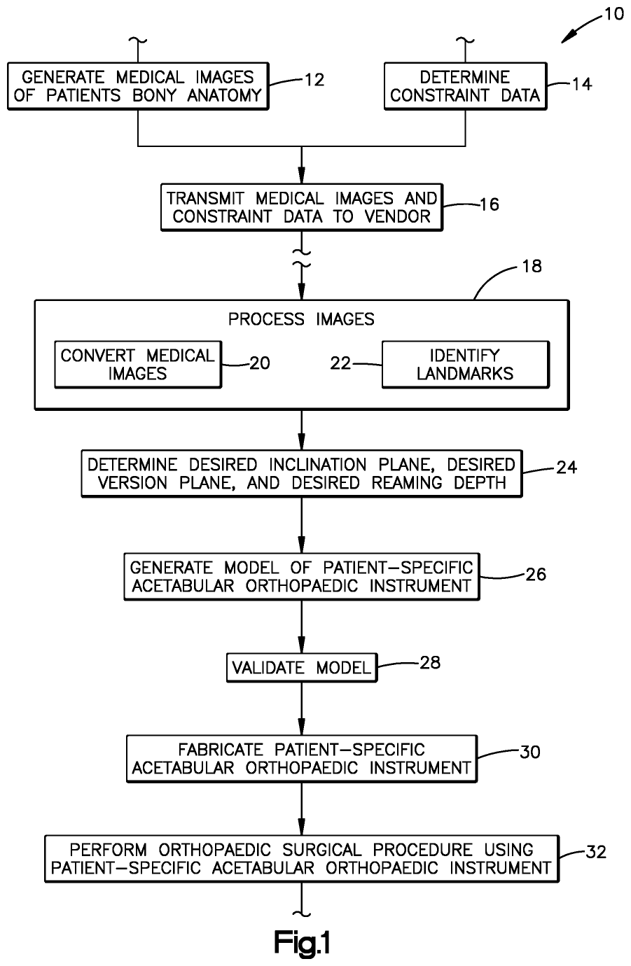

Referring to FIG. 1, a method 10 is provided for fabricating a customized patient-specific orthopaedic surgical instrument. The customized patient-specific orthopaedic surgical instrument can, in one example, be a surgical tool configured for use by a surgeon in performing an orthopaedic surgical procedure that is intended, and configured, for use on a particular patient. For instance, the surgical tool is customized to fit a specific anatomy of the particular patient, recognizing that the surgical tool does not fit an anatomy of other patients. As such, it should be appreciated that, as used herein, the term “customized patient-specific orthopaedic surgical instrument” is distinct from standard, non-patient specific orthopaedic surgical instruments that are intended for use on a variety of different patients. Additionally, it should be appreciated that, as used herein, the term “customized patient-specific orthopaedic surgical instrument” is distinct from orthopaedic prostheses, whether patient-specific or generic, which are surgically implanted in the body of the patient. Rather, customized patient-specific orthopaedic surgical instruments are used by an orthopaedic surgeon to assist in the implantation of orthopaedic prostheses.

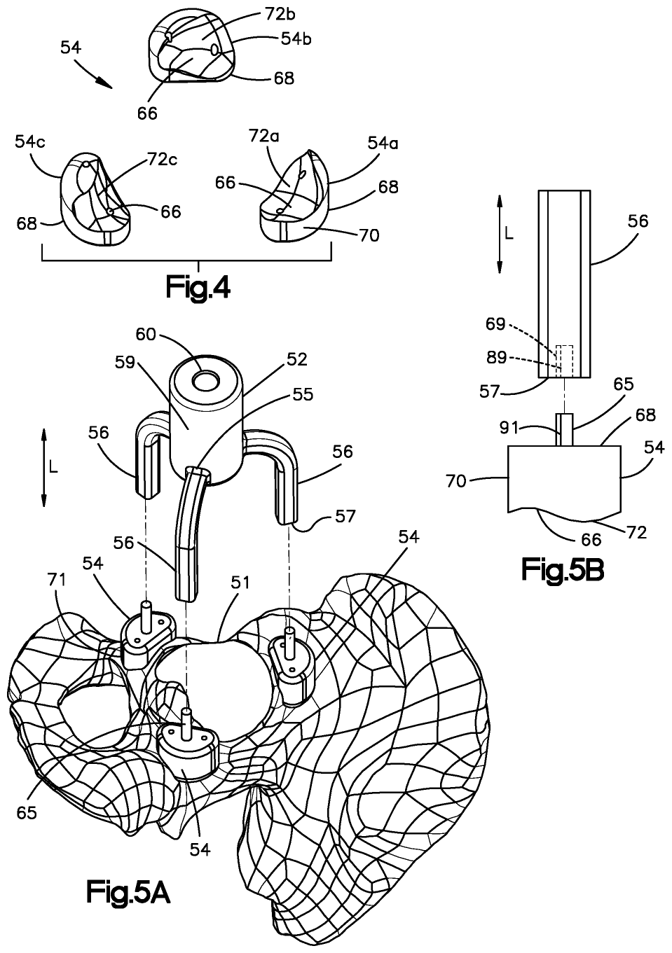

In some embodiments, the customized patient-specific acetabular orthopaedic surgical instrument may be customized to the particular patient based on the location at which the instrument is to be fit onto one or more bones of the patient. In one example, the customized patient-specific acetabular orthopaedic surgical instrument can be configured to fit onto at least a portion of the acetabulum. For example, in some embodiments, the customized patient-specific acetabular orthopaedic surgical instrument may include at least one bone-contacting or bone-facing surface having a positive contour that matches the contour of a portion of the underlying bone of the patient, which is discussed in more detail below in regard to FIG. 4. As such, the customized patient-specific acetabular orthopaedic surgical instrument is configured to be coupled to patient's coxal bone in a unique location and position with respect to the patient's bony anatomy. That is, the bone-contacting surfaces are configured to mate with matching inverse contours of the patient's coxal bone. As the coxal bone includes the ilium bone, the ischium bone, and the pubis bone, the bone-contacting surfaces can be configured to mate with matching inverse contours of at least one or more up to all of the ilium bone, the ischium bone, and the pubis bone. That is, the bone-contacting surfaces can include at least one positive contour that is are configured to receive a matching negative contour surface of the portion of the patient's coxal bone.

As such, the orthopaedic surgeon's guesswork and/or intra-operative decision-making with respect to the placement of the patient-specific acetabular orthopaedic surgical instrument are reduced. For example, the orthopaedic surgeon may not be required to locate landmarks of the patient's bone to facilitate the placement of the patient-specific acetabular orthopaedic surgical instrument, which typically requires some amount of estimation on part of the surgeon. Rather, the orthopaedic surgeon may simply locate the customized patient-specific acetabular orthopaedic surgical instrument to the patient's coxal bone in a unique location of the particular patient such that the contours of the at least one bone-contacting surface mate with the inverse contours of the patient's coxal bone. Further, the orthopaedic surgeon can couple the customized patient-specific acetabular orthopaedic surgical instrument to the patient's coxal bone in the unique location. When so coupled, the patient-specific acetabular orthopaedic surgical instrument defines a particular predetermined orientation with respect to planes of anteversion and inclination.

As shown in FIG. 1, the method 10 includes process steps 12 and 14, in which an orthopaedic surgeon performs pre-operative planning of the acetabular orthopaedic surgical procedure to be performed on a patient. The process steps 12 and 14 may be performed in any order or contemporaneously with each other. In process step 12, a number of medical images of a patient's bony anatomy are generated. The patient's bony anatomy can include one or both of the patient's acetabulum and the surrounding bony anatomy. To do so, the orthopaedic surgeon or other healthcare provider may operate an imaging system to generate the medical images. The medical images may be embodied as any number and type of medical images capable of being used to generate a three-dimensional rendered model of the patient's acetabulum and surrounding bony anatomy. For example, the medical images may be embodied as any number of computed tomography (CT) images, magnetic resonance imaging (MM) images, or other three-dimensional medical images. Additionally, or alternatively, as discussed in more detail below in regard to process step 18, the medical images may be embodied as a number of X-ray images or other two-dimensional images from which a three-dimensional rendered model of the area of the patient's coxal bone proximate to the acetabulum and the surrounding bony anatomy may be generated. The medical images can further include information regarding bone density of the patient's acetabulum and the surrounding bony anatomy.

In process step 14, the orthopaedic surgeon may determine any additional pre-operative constraint data. The constraint data may be based on the orthopaedic surgeon's preferences, preferences of the patient, anatomical aspects of the patient, guidelines established by the healthcare facility, or the like. For example, the constraint data may include the orthopaedic surgeon's preference for the amount of inclination and anteversion for the acetabular prosthesis, the amount of the bone to ream, the size range of the orthopaedic implant, and/or the like. In some embodiments, the orthopaedic surgeon's preferences are saved as a surgeon's profile, which may be used as a default constraint values for further surgical plans.

In process step 16, the medical images and the constraint data, if any, are transmitted or otherwise provided to an orthopaedic surgical instrument vendor or manufacturer. The medical images and the constraint data may be transmitted to the vendor via electronic means such as a network or the like. Thus, the process step 16 can also be referred to as a step of receiving the medical images and the constraint data. After the vendor has received the medical images and the constraint data, the vendor processes the images in step 18. The orthopaedic surgical instrument vendor or manufacturer process the medical images to facilitate the determination of the proper planes of inclination and anteversion, implant sizing, and fabrication of the customized patient-specific acetabular orthopaedic surgical instrument as discussed in more detail below.

In process step 20, the vendor may convert or otherwise generate three-dimensional images from the medical images. For example, in embodiments wherein the medical images are embodied as a number of two-dimensional images, the vendor may use a suitable computer algorithm to generate one or more three-dimensional images form the number of two-dimensional images. Additionally, in some embodiments, the medical images may be generated based on an established standard such as the Digital Imaging and Communications in Medicine (DICOM) standard. In such embodiments, an edge-detection, thresholding, watershed, or shape-matching algorithm may be used to convert or reconstruct images to a format acceptable in a computer aided design application or other image processing application.

In process step 22, the vendor may process the medical images, and/or the converted/reconstructed images from process step 20, to determine a number of aspects related to the bony anatomy of the patient such as the anatomical axis of the patient's bones, the mechanical axis of the patient's bone, other axes and various landmarks, bone density, and/or other aspects of the patient's bony anatomy. To do so, the vendor may use any suitable algorithm to process the images.

In process step 24, the desired inclination and anteversion planes for implantation of the acetabular orthopaedic prosthesis are determined. The planned inclination and anteversion planes may be determined based on the type, size, and position of the acetabular orthopaedic prosthesis to be used during the orthopaedic surgical procedure; the process images, such as specific landmarks identified in the images; and the constraint data supplied by the orthopaedic surgeon in process steps 14 and 16. The type and/or size of the acetabular orthopaedic prosthesis may be determined based on the patient's anatomy and the constraint data. For example, the constraint data may dictate the type, make, model, size, or other characteristic of the acetabular orthopaedic prosthesis. The selection of the acetabular orthopaedic prosthesis may also be modified based on the medical images such that an acetabular orthopaedic prosthesis that is usable with the acetabulum of the patient and that matches the constraint data or preferences of the orthopaedic surgeon is selected.

In addition to or as an alternative to the type and size of the acetabular orthopaedic prosthesis, the planned location and position of the acetabular orthopaedic prosthesis relative to the patient's bony anatomy is determined. To do so, a digital template of the acetabular orthopaedic prosthesis may be overlaid onto one or more of the processed medical images. The vendor may use any suitable algorithm to determine a recommended location and orientation of the acetabular orthopaedic prosthesis (i.e., the digital template) with respect to the patient's bone based on the processed medical images (e.g., landmarks of the patient's acetabulum defined in the images) and/or the constraint data. Additionally, any one or more other aspects of the patient's bony anatomy may be used to determine the proper positioning of the digital template.

In some embodiments, the digital template along with surgical alignment parameters may be presented to the orthopaedic surgeon for approval. The approval document may include the implant's planned inclination and anteversion planes, the orientation of the transverse acetabular ligament and labrum, and other relevant landmarks of the patient's bony anatomy.

The proper inclination and anteversion planes for the acetabular orthopaedic prosthesis may then be determined based on the determined size, location, and orientation of the acetabular orthopaedic prosthesis. In addition, other aspects of the patient's bony anatomy, as determined in process step 22, may be used to determine or adjust the planned inclination and anteversion planes. For example, the determined mechanical axis, landmarks, and/or other determined aspects of the relevant bones of the patient may be used to determine the planned inclination and anteversion planes.

In process step 26, a model of the customized patient-specific acetabular orthopaedic surgical instrument is generated. In some embodiments, the model is embodied as a three-dimensional rendering of the customized patient-specific acetabular orthopaedic surgical instrument. In other embodiments, the model may be embodied as a mock-up or fast prototype of the customized patient-specific acetabular orthopaedic surgical instrument. The patient-specific acetabular orthopaedic surgical instrument to be modeled and fabricated may be determined based on the acetabular orthopaedic surgical procedure to be performed, the constraint data, and/or the type of orthopaedic prosthesis to be implanted in the patient.

The particular shape of the customized patient-specific acetabular orthopaedic surgical instrument is determined based on the planned location and implantation angles of the acetabular orthopaedic prosthesis relative to the patient's acetabulum. The planned location of the customized patient-specific acetabular orthopaedic surgical instrument relative to the patient's acetabulum may be selected based on, in part, the planned inclination and anteversion planes of the patient's acetabulum as determined in step 24. Further, if desired, the planned location of the customized patient-specific acetabular orthopaedic surgical instrument relative to the patient's acetabulum may also be selected based on the bone density of the patient's acetabulum and surrounding bony anatomy. For example, in some embodiments, the customized patient-specific acetabular orthopaedic surgical instrument is embodied as an acetabular guide assembly. In such embodiments, the location of the acetabular guide assembly is configured to provide an acetabular reamer guide assembly that, in turn, is configured to position the acetabular orthopaedic prosthesis at the planned inclination and anteversion planes determined in process step 24. Additionally, the planned location of the orthopaedic surgical instrument may be based on the identified landmarks of the patient's acetabulum identified in process step 22. Further still, the planned location of the orthopaedic surgical instrument can be based on the bone density of the acetabulum. In this regard, it is recognized that the orthopaedic surgical instrument can be fastened to the acetabulum or surrounding bony anatomy. It can be desirable to couple the orthopaedic surgical instrument to regions of sufficient bone density.

In some embodiments, the particular shape or configuration of the customized patient-specific acetabular orthopaedic surgical instrument may be determined based on the planned location of the instrument relative to the patient's bony anatomy. That is, the customized patient-specific acetabular orthopaedic surgical instrument may include at least one bone-contacting surface having a contour that matches a corresponding inverse contour of a portion of the bony anatomy of the patient such that the orthopaedic surgical instrument may be fitted onto the bony anatomy of the patient in a unique location, which corresponds to the pre-planned location for the instrument. For instance, a three dimensional model of an orthopaedic surgical instrument can be positioned such that a portion of the instrument overlies the three-dimensional model of the underlying coxal bone at a predetermined specific location. Thus, the intersection of the surface of the underlying coxal bone and the model of the instrument can define a bone-facing or bottom surface of the instrument. Thus, the bottom surface of the instrument, when manufactured, can be contoured to fit onto the specific location of the patient's underlying coxal bone. The instrument can be configured as one or more mounting pads for an acetabular guide assembly as described below, Further, the at least one bone-contacting surface can receive a fastener that extends into the bony anatomy of the patient to temporarily couple the orthopaedic surgical instrument to the bony anatomy. When the orthopaedic surgical instrument is coupled to the patient's bony anatomy in the unique location and at a desired orientation, one or more guides (e.g., cutting or drilling guide) of the orthopaedic surgical instrument may be aligned to the inclination and anteversion planes, as discussed above.

After the model of the customized patient-specific acetabular orthopaedic surgical instrument has been generated in process step 26, the model is validated in process step 28. The model may be validated by, for example, analyzing the rendered model while coupled to the three-dimensional model of the patient's anatomy to verify the correlation of reaming guides, inclination and anteversion planes, and/or the like. Additionally, the model may be validated by transmitting or otherwise providing the model generated in step 26 to the orthopaedic surgeon for review. For example, in embodiments wherein the model is a three-dimensional rendered model, the model along with the three-dimensional images of the patient's acetabulum and area of the coxal bone proximate to the acetabulum may be transmitted to the surgeon for review. In embodiments wherein the model is a physical prototype, the model may be shipped to the orthopaedic surgeon for validation.

After the model has been validated in process step 28, the customized patient-specific acetabular orthopaedic surgical instrument is fabricated in process step 30. For instance, manufacturing instructions can be generated to fabricate the customized patient-specific acetabular orthopaedic surgical instrument at a remote location by a third party. Alternatively, the customized patient-specific acetabular orthopaedic surgical instrument can be fabricated on site. Advantageously, at least a portion up to an entirety of the customized patient-specific acetabular orthopaedic surgical instrument may be fabricated using any suitable additive manufacturing process. Additionally, the customized patient-specific acetabular orthopaedic instrument may be formed from any suitable material such as a metallic material, a plastic material, or combination thereof depending on, for example, the intended use of the instrument. The fabricated customized patient-specific acetabular orthopaedic instrument is subsequently shipped or otherwise provided to the orthopaedic surgeon. The surgeon performs the orthopaedic surgical procedure in process step 32 using the customized patient-specific acetabular orthopaedic surgical instrument. As discussed above, because the orthopaedic surgeon does not need to determine the proper location of the orthopaedic surgical instrument intra-operatively, which typically requires some amount of estimation on part of the surgeon, the guesswork and/or intra-operative decision-making on part of the orthopaedic surgeon is reduced.

It should also be appreciated that variations in the bony of anatomy of the patient may require more than one customized patient-specific acetabular orthopaedic surgical instrument to be fabricated according to the method described herein. For example, the patient may require the implantation of two acetabular orthopaedic prostheses to replace both natural hips. As such, the surgeon may follow the method 10 of FIG. 1 to fabricate a different customized patient-specific acetabular orthopaedic surgical instrument for use in replacing each natural hip. Each customized patient-specific acetabular orthopaedic surgical instrument defines a particular degree of anteversion and inclination angles relative to each particular acetabulum that is different due to the variation in the bony anatomy of each hip.

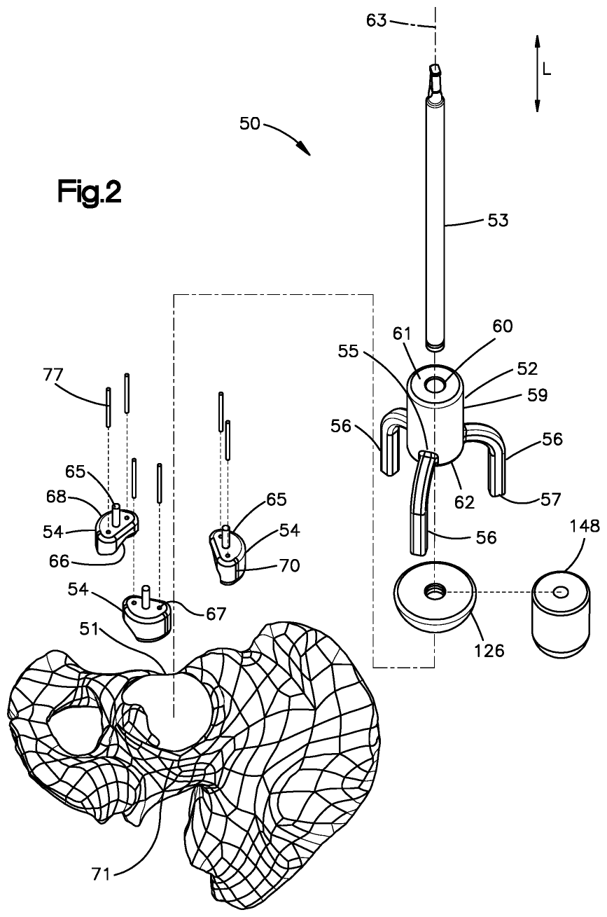

Referring now to FIG. 2, in one embodiment, the customized patient-specific acetabular orthopaedic surgical instrument may be embodied as an acetabular guide assembly 50. In one example, the acetabular guide assembly 50 is usable by a surgeon to guide a reamer 126 to the patient's acetabulum 51 in a predetermined location and orientation that will position the acetabular orthopaedic prosthesis at the desired, predetermined angles of inclination and anteversion. For instance, the guide member 52 has a passageway 60 that receives a tool shaft 53 of the acetabular guide assembly 50. The tool shaft 53 is couplable to a reamer 126, and orients the reamer 126 at the predetermined location and orientation. Alternatively, the passageway 60 can receive a bone guide pin and guide the bone guide pin to be secured to the patient's acetabulum 51 in a predetermined location and orientation that will position the acetabular orthopaedic prosthesis at the desired, predetermined angles of inclination and anteversion. The bone guide pin can then be subsequently used to orient and guide a cannulated reamer. In this regard, the acetabular guide assembly 50 can be referred to as an acetabular drill guide assembly. The bone guide pin can further be used to orient and guide an impactor 148 as discussed in more detail below. Thus, the acetabular guide assembly 50 can alternatively or additionally be referred to as an acetabular impactor guide assembly. It is further appreciated that an acetabular implantation system can include the acetabular guide assembly 50, either or both of the reamer and the impactor, and can further include the acetabular orthopaedic prosthesis.

The illustrated acetabular guide assembly 50 includes a guide member 52, a plurality of arms 56, and a plurality of mounting pads 54. The arms 56 can extend from the guide member 52 to respective ones of the mounting pads 54. Accordingly, the arms 56 can fixedly support the guide member 52 at a predetermined position and orientation relative to the mounting pads 54. The guide member 52 includes a guide body 58 and a longitudinal passageway 60 that extends through guide body 58 from a top surface 61 of the guide body 59 to a bottom surface 62 of the guide body 59 along a central axis 63 that is oriented along a longitudinal direction L. The guide body 58 can have a substantially cylindrical shape in one example. The central axis 63 can be oriented parallel to or can be coincident with the central axis of the cylindrical guide body 58. It should be appreciated that the central axis 63 can be alternatively oriented as desired. Further, the guide body 58 can have other shapes in other embodiment or examples of the design. For instance, the guide body 58 may have a substantially rectangular, triangular, polygonal cross-section, or any suitable alternative cross-section. The cross-section can be taken along a plane that is oriented perpendicular to the longitudinal direction L.

The acetabular guide assembly 50 is configured to receive the tool shaft 53 in the longitudinal passageway 60, such that the tool shaft 53 that is movable in the longitudinal passageway 60. For instance, the tool shaft 53 can be translatable and rotatable in the longitudinal passageway 60 when the guide member 52 provides both a drill guide and an impactor guide. In other embodiments, the guide member can be configured as an impactor guide, but not as a drill guide. Thus, the longitudinal passageway 60 can permit translation of the tool shaft 53 but can prevent rotation of the tool shaft 53. In one example, the passageway 60 can have a substantially circular cross-section, or can define any suitable alternative cross-sectional shape as desired. It is recognized that the tool shaft 53 can be inserted directly into the passageway. Alternatively, the passageway 60 can retain a sleeve that, in turn, translatably and/or rotatably receives the tool shaft 53.

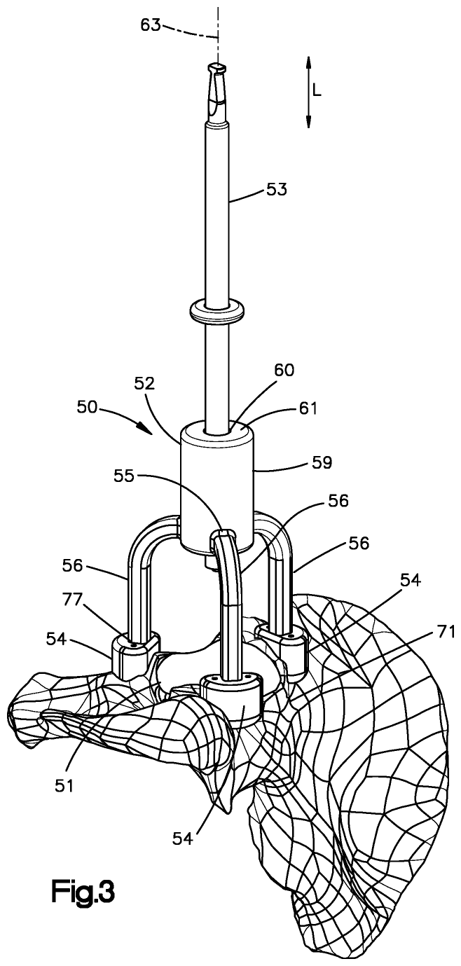

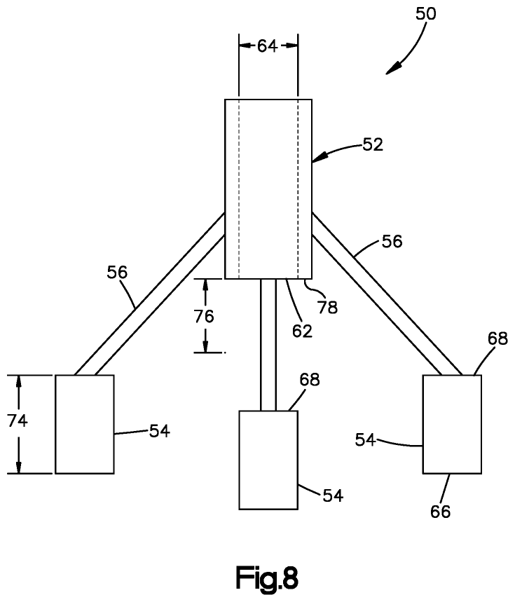

As used herein, the term “substantially” and derivatives thereof, and words of similar import, when used to describe a size, shape, orientation, distance, spatial relationship, or other parameter includes the stated size, shape, orientation, distance, spatial relationship, or other parameter, and can also include a range up to 10% more and up to 10% less than the stated parameter, including 5% more and 5% less, including 3% more and 3% less, including 1% more and 1% less. As illustrated in FIG. 3, the passageway 60 may have a cross-sectional dimension 64 (see FIG. 8) that is slightly larger than the cross-sectional dimension of the tool shaft 53, such that the passageway 60 defines a guide for movement of the tool shaft 53 therein. The passageway 60 and the tool shaft 53 can be cylindrical, such that the cross-sectional dimensions 64 of the passageway 60 and the cross-sectional dimension of the tool shaft 53 can be defined by diameters. It is recognized, however, that the passageway 60 and the tool shaft 53 can have any suitable cross-sectional shape and dimension as desired. The tool shaft 53 can be configured to be coupled to a reamer 126. Thus, the tool shaft 53 can be rotatable in the longitudinal passageway 60 about the central axis 63. Alternatively, the shaft can be configured to be coupled to an impactor. Thus, the shaft can be translatable along the central axis 63 so as to impact an acetabular orthopaedic prosthesis so as to drive the prosthesis into the underlying bone. In some examples, the shaft can be selectively coupled to a reamer and an impactor.

In still other examples, the passageway 60 can be to receive a bone guide pin so as to allow the guide pin to be secured to the patient's underlying acetabulum 51. For example, the passageway 60 can have a substantially circular cross-section. In other embodiments, the guide body 58 may include a passageway 60 configured to receive a guide pin with a different cross-sectional shape.

Referring now also to FIG. 3, each of the mounting pads 54 is configured to contact the patient's bony anatomy during use. For instance, each of the mounting pads 54 can be individually contoured to fitted onto respective different and separate unique locations of the coxal bone 71. When the mounting pads are fitted onto the respective different locations of the coxal bone 71 and the guide member 52 is supported relative to the mounting pads 54, the guide member 52 can be similarly supported relative to the underlying coxal bone. As will be appreciated from the description below, the guide member 52 can be supported at a predetermined orientation with respect to the planes of inclination and anteversion.

In particular, referring now to FIG. 4, each of the mounting pads 54 includes a bottom surface 66, which is configured to contact a portion of the area of the patient's coxal bone 71 proximate to the acetabulum 51. Each mounting pad 54 also includes a top surface 68 opposite the bottom surface 66 along the longitudinal direction L, and a sidewall 70 that extends from the top surface 68 to the bottom surface 66. As discussed in greater detail below, the position of each mounting pad 54 relative to the guide member 52 and relative to each other allows the acetabular guide assembly 50 to be coupled to the patient's coxal bone 71 in a predetermined orientation and location relative to the acetabulum 51.

The bottom surface 66 of each mounting pad 54 may be customized to the contour of the patient's coxal bone 71. For example, the bottom surfaces 66 of the mounting pads 54 are configured with a customized patient-specific contour 72 configured to mate with a portion of the corresponding contour of the patient's coxal bone 71 proximate to the acetabulum 51. In one example, the patient specific-contour 72 can include at least one positive contour that is configured to receive a corresponding at least one negative contour of the underlying coxal bone 71. In one example, the acetabular guide assembly 50 can include a plurality of mounting pads 54 that are each configured to be fitted onto a single coxal bone 71 that is disposed proximate to a single acetabulum. In one example, the mounting pads 54 can be positioned about the acetabulum 51 such that the bottom surfaces 66 are fitted onto the coxal bone 71 so as to mate with the coxal bone 71. Further, the bottom surfaces 66 can extend to the acetabular rim, such that they mate with a portion of the acetabular rim.

While the acetabular guide assembly 50 is illustrated as including three mounting pads 54a-54c, it should be appreciated that the acetabular guide assembly 50 can include any number of mounting pads as desired, including at least one mounting pad. The patient-specific contours 72 of the bottom surface 66 of each of the mounting pads 54 can be different than the contours of the bottom surface 66 of all others of the mounting pads 54. Each of the mounting pads 54a-54c can include a respective patient-specific contour 72a-c that are all different than each other. Thus, the patient specific contour 72a of the first mounting pad 54a is configured to mate with the coxal bone 71 at a first unique location. The patient specific contour 72b of the second mounting pad 54b is different than the patient specific contour 72a of the first mounting pad 54a, and is configured to mate with the coxal bone 71 at a second unique location different than the first unique location. The patient specific contour 72c of the third mounting pad 54c is different than the patient specific contours 72a and 72b of the first and second mounting pads 54a and 54b, respectively, and is configured to mate with the coxal bone 71 at a third unique location different than the first and second unique locations. In one example, at least a respective portion of the first, second, and third unique locations can be spaced from each other along a circumference of a circle when viewed from a top plan view. It is recognized, however, that the first, second, and third unique locations can be disposed in any alternative arrangement suitable to support the acetabular guide assembly 50 in the manner described herein.

As such, referring to FIGS. 5A-5B, the acetabular guide assembly 50 is configured to be coupled to the patient's coxal bone 71 in a desired position and orientation, which has been predetermined to establish a desired location and orientation of the central axis 63 of the passageway 60, which in turn can determine the inclination and anteversion planes of the acetabular orthopaedic prosthesis. For instance, each mounting pad 54 of the plurality of mounting pads 54 can be configured to couple to a respective arm 56 of the plurality of arms 56 at a respective datum. That is, the mounting pads 54 can include respective coupling members configured to couple to corresponding coupling members of the plurality of arms 56, respectively, at a datum. The datum can be a fixed datum in some examples. In one example, the coupling member of the mounting pads can include a boss 65 that extends out from an outer surface of the mounting pad 54. In one example, the outer surface can be defined by the top surface 68. For instance, the boss 65 can extend out from the top surface 68 along the longitudinal direction L. Thus, when the top surface 68 is planar, the boss 65 can extend out from the top surface 68 along a direction that is perpendicular to the top surface 68. Each arm 56 of the plurality of arms 56 can define a proximal end 55 that is coupled to or otherwise extends from the guide member 52, and a distal end 57 opposite the proximal end 55.

The coupling member of each of the arms 56 can be configured as an opening 69 that extends into the distal end 57. The opening 69 can be sized to receive the boss 65 of the respective mounting pad 54 so as to couple the arms 56 to the respective mounting pads 54. Thus, the arms 56 can be brought toward the mounting pads 54 until the bosses 65 are received in the openings 69, respectively, thereby coupling the arms 56 to the mounting pads 54. In one example, the arms 56 can be brought toward the mounting pads 54 along the longitudinal direction L until the bosses 65 are received in the openings 69, respectively. The arms 56 can be positionally fixed with respect to either or both of the guide member 52 and the mounting pads 54 in one example.

If the depth of the opening 69 is less than the length of the boss 65, the datum can be defined at the distal tip of the boss 65 that abuts the arm 56 in the opening 69. Alternatively, if the depth of the opening 69 is greater than the length of the boss 65, the datum can be defined at the top surface 68 of the mounting pad 54 that abuts the distal end of the arm 56. If the depth of the opening 69 is substantially equal to the length of the boss then the datum can be defined by both the distal tip of the bass 65 and the top surface 68 of the mounting pad. Thus, in all examples, each of the mounting pads 54 can define a datum. In one example, the opening 69 can extend into the distal end 57 along the longitudinal direction L. Thus, movement of the arms 56 toward the mounting pads 54 along the longitudinal direction L will cause the bosses 65 to be received in the respective openings 69, thereby coupling the arms 56 to the mounting pads 54. It should be appreciated, of course, that the arms 56 and the at least one mounting pad 54 can define any alternative coupling structure suitable to couple the arms 56 to the at least one mounting pad 54. For instance, the arms 56 can define a projection that is received by an aperture that extends into an outer surface of the at least one mounting pad 54.

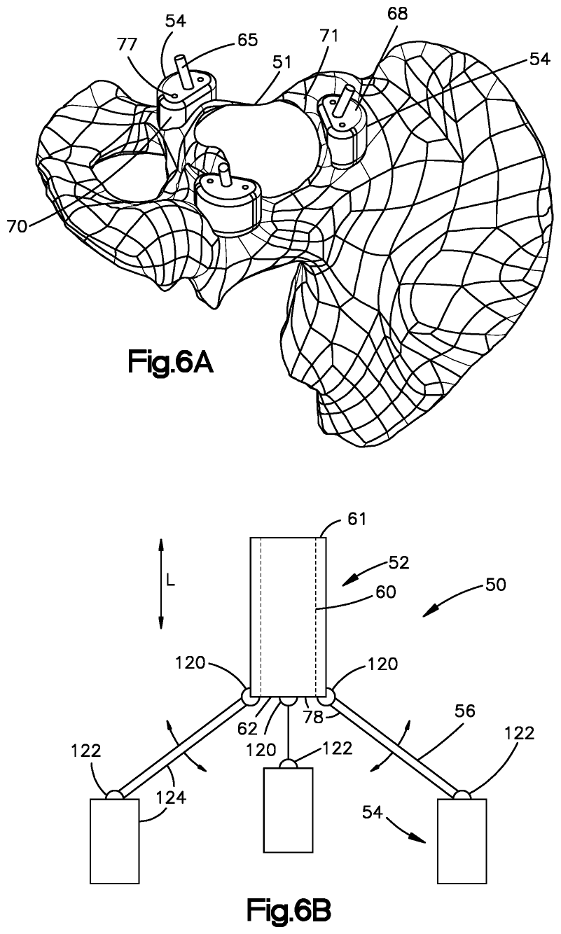

Referring now to FIGS. 6A-6B, in another example, at least one or more of the bosses 65 up to all of the bosses 65 can extend out from any suitable surface of the mounting pads 54 along a direction that is angularly offset with respect to the longitudinal direction L. In the illustrated embodiment, at least one or more of the bosses 65 up to all of the bosses can extend from the top surface 68. Alternatively, at least one or more of the bosses 65 up to all of the basses can extend out from the side surface 70.

Further, each arm 56 can be moveably coupled to either or both of the guide member 52 and the respective mounting pad 54. In particular, each arm 56 can be secured to a joint 120 of the guide member 52 and a corresponding joint 122 of the respective mounting pad 54. The joints 120 and 122 may be configured as hinges, universal joints, or the like configured to allow the openings 69 of the arms 56 to receive the bosses 65 of the mounting pads 54 when the bosses 65 are oriented angularly offset with respect to the longitudinal direction L. For instance, the openings 69 of the arms 56 can be aligned with the respective bosses 65, and then moved toward the mounting pads 54 until the bosses 65 are received in the openings 69. The joints 120 and 122 can further include a locking mechanism if desired that is capable of fixing the respective arm 56 at a desired position. For instance, the locking mechanism can fix the arm 56 at a position coupled to each of the guide member 52 and the respective mounting pad 54 while the guide member is in the predetermined position and orientation. It will be appreciated that in other embodiments not all arms 56 may be moveably secured to the guide member 52 and/or mounting pads 54. Additionally, the acetabular guide assembly 50 may include any combination of joints to position the acetabular guide assembly 50 at the planned orientation and location to establish the desired inclination and anteversion planes of the acetabular orthopaedic prosthesis.

It is further recognized that the arms 56 can be coupled to the mounting pads 54 in accordance with any suitable alternative embodiment. For instance, the mounting pads 54 can define an opening 69 that extends into the top surface 68 and is sized to receive the distal ends 57 of the respective mounting pads 54 so as to couple the arms 56 to the mounting pads 54 at the datum. The arms 56 can be coupled to respective ones of the mounting pads 54 via suitable fasteners such as screws, bolts, adhesive, or the like.

In the illustrative embodiment, each datum has a predetermined spatial relationship with respect to other datums when the mounting pads 54 are fitted onto their respective unique positions at the coxal bone 71 such that the bottom surfaces 56 mate with the coxal bone 71 in the manner described above. In one example, the datum can be substantially coplanar with each other in a plane that defines a predetermined angular relationship with the plane of inclination and the anteversion plane. In one example, the plane can be oriented substantially parallel with the anteversion plane. Alternatively, the plane can define a predetermined angle with respect to the anteversion plane. The arms 56 can all have substantially the same length from the guide member 52 to the mounting pads 54. The guide member 52 can be configured such that the central axis 63 of the passageway 60 is oriented to the plane defined by the datum when the arms 56 extend out from the guide member 52 and are coupled to the mounting pads 54.

As described above, the mounting pads 54 are configured to be fitted onto different unique locations of the underlying coxal bone 71. Further, it is recognized that the unique locations of the underlying coxal bone 71 may be non-planar with each other. Accordingly, one or more of the mounting pads 54 can define different thicknesses along the longitudinal direction L from the top surface 68 to the bottom surface 66 with respect to one or more others of the mounting pads, such that the bosses 65 that extend out from the top surface 68 can be substantially coplanar with each other in the manner described above.

Accordingly, the arms 56 and guide members 52 do not need to be customized, but rather can be used in combination with multiple kits of mounting pads 54. For instance, the guide member 52 and arms 56 can be pre-fabricated such that the central axis 63 of the passageway 60 is oriented normal to a plane along which the arms 56 are configured to attach to the mounting pads 54. Thus, if it is desired to orient the central axis 63 of the passageway 60 perpendicular to the anteversion plane, the same guide member 52 and arms 56 can be coupled to respective different kits of mounting pads 54 whose datum lie substantially in the plane that is substantially parallel to the anteversion plane when fitted onto the coxal bone 71. In this example, each kit of mounting pads 54 includes at least one mounting pad 54 configured to couple to the arms 56 so as to support a guide member 52 at a desired position and orientation as described herein. The at least one bottom surface of the at least one mounting pad 54 of each kit of mounting pads 54 can be individually and uniquely contoured as described above, but can also define datum that have the same relative positions and orientations. Alternatively, as described below, the arms 56 can be movable with respect to the guide member 52 so as to attach to the datum of respective kits of mounting pads 54 whose datum are disposed at different relative positions and orientations. As a result, the acetabular guide assembly 50 can be customized by customizing only the mounting pads 54. The guide member 52 and the arms 56 can be sterilized and reused as desired.

As a result, referring again to FIG. 2, a plurality of acetabular guide assemblies 50 can include substantially identical guide members 52 and different mounting pads 54. The term “substantially identical” and derivatives thereof as used herein refer to being designed to be identical and within manufacturing tolerances. Thus, the term “different” when used in connection with a comparison to different sizes, orientations, angles, shapes, or other value means that the compared values are different than each other by design, and thus outside of manufacturing tolerances. Because the guide members 52 of the acetabular guide assemblies 50 can be substantially identical to each other, they may be referred to as generic guide members 52. The mounting pads 54 of the guide assemblies can have respective contours 72 that are different than the contours of the mounting pads 54 of the other acetabular guide assemblies 50. Accordingly, the guide members 52 of the acetabular guide assemblies 50 can be supported by the respective mounting pads 54 at the patient-specific predetermined locations and orientations. In some examples, the arms 56 of the acetabular guide assemblies 50 can be substantially identical to each other, and thus may be referred to as generic arms 56. Alternatively, the arms 56 of the acetabular guide assemblies 50 can be sized and/or shaped differently from each other as desired.

It is therefore recognized that a plurality of different sets of mounting pads 54 can be produced. Each set of mounting pads 54 can be included in a different acetabular guide assembly 50 of the plurality of acetabular guide assemblies 50. The contours 72 of the mounting pads 54 of the respective sets of mounting pads 54 are configured to match, or be fitted to, respective contours of different coxal bones at unique locations of the coxal bones. The different coxal bones are defined by different patients. Further, each set of mounting pads 54 is configured to support substantially identical generic guide members 52 at a desired orientation with respect to the angles of inclination and anteversion of the respective patient. Thus, a plurality of acetabular guide assemblies 50 can be constructed using substantially identical guide members 52, different mounting pads 54, and either substantially identical arms 56 or different arms 56 to support the guide members 52 at a patient-specific orientation with respect to the angles of inclination and anteversion.

In one example, the sets of mounting pads 54 can be produced non-contemporaneously. That is, the sets of mounting pads 54 can be produced on a patient-by-patient basis at different times. For instance, the sets of mounting pads 54 can be produced days, weeks, months or even years apart. Further, the sets of mounting pads 54 can be packaged and delivered separately to different healthcare providers. Therefore, it is recognized that sets of mounting pads 54 can be produced that are not provided in a single kit. In other examples, it is recognized that sets of mounting pads 54 described herein can be provided in a kit, such that a healthcare provider can have an inventory of the mounting pads 54 with different contours 72.

In this regard, first and second acetabular guide assemblies 50 can each include a guide body 52 that defines the longitudinal passageway 60. The guide body 52 of the first acetabular guide assembly 50 is substantially identical to the guide body 52 of the second acetabular guide assembly 50. Each of the first and second acetabular guide assemblies 50 further include a plurality of additively manufactured mounting pads 54 each having respective patient-specific positive contours 72 that match corresponding negative contoured surfaces at unique locations of a coxal bone proximate to an acetabulum. The patient-specific positive contours 72 of the mounting pads 54 of the first acetabular guide assembly 50 are all different than the patient-specific positive contours 72 of the mounting pads 54 of the second acetabular guide assembly 50. Each of the first and second acetabular guide assemblies 50 includes a plurality of arms 56 configured to extend from the guide body 52 to the plurality of mounting pads 54, wherein the arms 56 of the first acetabular guide assembly 50 are configured to support the guide body 52 of the first acetabular guide assembly 50 at a first predetermined location and orientation with respect to the acetabulum of a first patient. The arms 56 of the second acetabular guide assembly 50 are configured to support the guide body 52 of the second acetabular guide assembly 50 at a second predetermined location and orientation with respect to the acetabulum of a second patient.

Further, as described below with reference to FIG. 5B, each of the mounting pads 54 of the first acetabular guide assembly 50 can be uniquely keyed to a corresponding one of the arms 56 of the first acetabular guide assembly 50 so as to be located at a first predetermined location and oriented in a first predetermined orientation with respect to the acetabulum of the first patient. Similarly, each of the mounting pads 54 of the second acetabular guide assembly 50 can be uniquely keyed to a corresponding one of the arms 56 of the second acetabular guide assembly 50 so as to be located at a second predetermined location and oriented in a second predetermined orientation with respect to the acetabulum of the second patient.

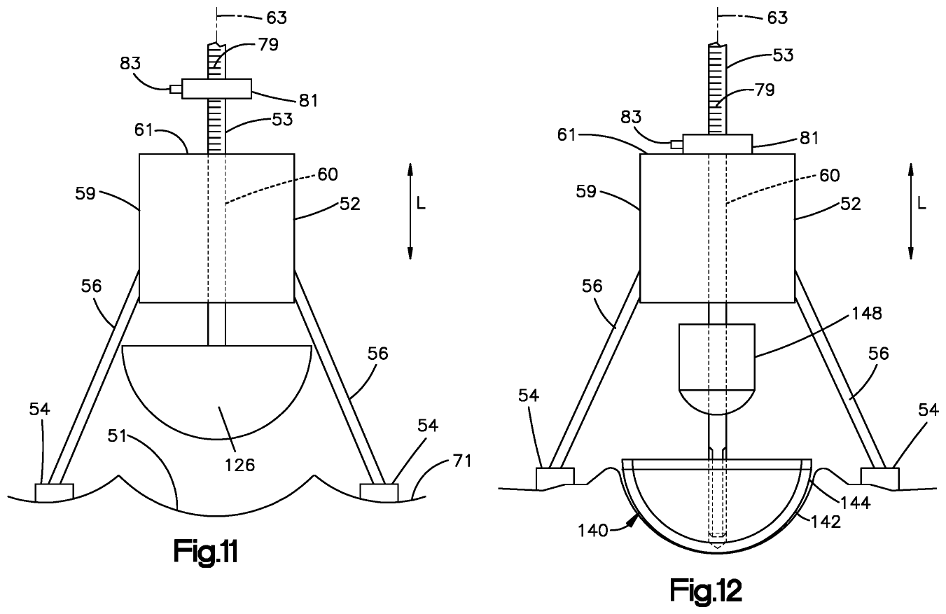

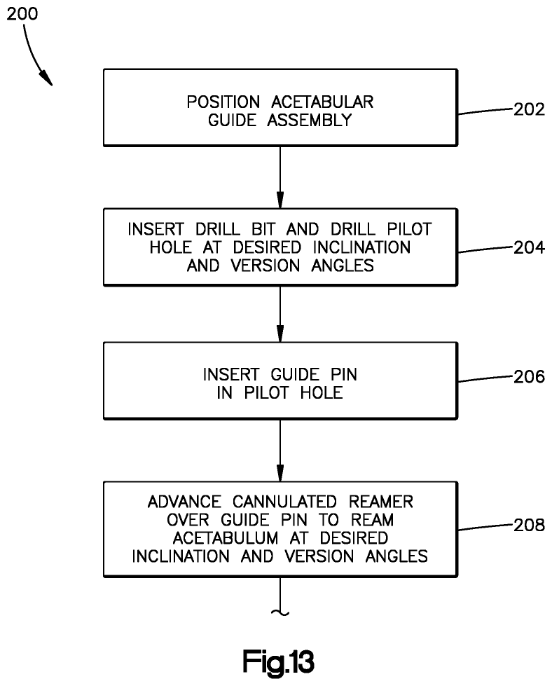

Further still, each of the first and second acetabular guide assemblies can include a tool shaft 53 configured to rotate and translate in the passageway 60 of the guide body 52. The tool shaft 53 is configured to selectively couple to a reamer and an impactor. As described in more detail below, the tool shaft can include a stop member 81 (see FIGS. 11-12) configured to abut the guide body so as to limit translation of the tool shaft 53 in the passageway 60.

With continuing reference to FIG. 2, the mounting pads 54 can be configured to be coupled to the respective unique locations of the underlying coxal bone 71 to which the mounting pads 54 are fitted. For instance, one or more bone fasteners 77 can be driven through the mounting pads 54 and into the underlying bone. In one example, the mounting pads 54 can define at least one fixation aperture 67 that extends therethrough. The fixation aperture 67 can be sized to receive a respective fastener 77 of the plurality of fasteners 77. The fixation aperture 67 can extend from the top surface 68 to the bottom surface 66. The fixation aperture 67 can be positioned and oriented along any suitable direction as determined during the method step 26 described above with respect to FIG. 1. For instance, the fixation aperture 67 can be positioned and oriented such that the fastener 77 received therein is directed to be driven into a desirable location of the underlying bone. Thus, the fixation aperture 67 can be oriented along the longitudinal direction L in one example. Alternatively, the fixation aperture 67 can be oriented along any suitable direction that is angularly offset with respect to the longitudinal direction L. In the illustrated, embodiment, each of the mounting pads 54 can include a pair of fixation apertures 67 disposed on opposite sides of the boss 65, each configured to receive a respective one of the plurality of fasteners in one example.

Each of the mounting pads 54 has a length, which may be determined based on the surface contour of the patient's bony anatomy such that the acetabular guide assembly 50 is positioned at the desired predetermined angles of inclination and anteversion. The length of each of the mounting pads 54 can be defined by a longest dimension of the mounting pad 54 that extends along the underlying coxal bone 71. In one example, the length of each mounting pad 54 can be substantially equal to one another. In other embodiments, the length of at least one of the mounting pads 54 can be different than the length of at least one other one of the mounting pads 54 depending, for instance, on the contours of the unique locations of the underlying bone. The unique locations can be selected to ensure that the mounting pads and arms 56 define a stable construct for the guide member 52. Further, the locations can be selected based on the porosity of the underlying bone. As the mounting pads 54 can further be coupled to the underlying bone, it may be desirable for the bone to be healthy bone. In this regard, of the mounting pads 54 can have any size and shape suitable to fit over the respective unique locations of the underlying coxal bone 71, define a stable base for the arms 56 and the guide member 52, and to receive the fasteners 77 that are driven into the underlying coxal bone 71.

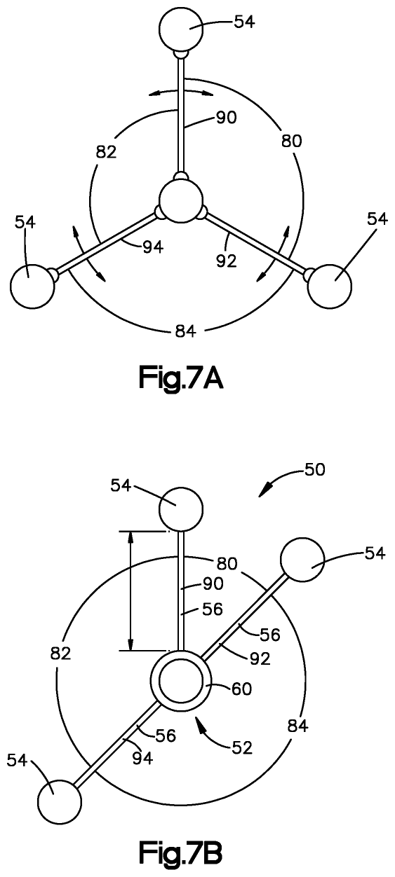

Referring now to FIG. 7A, the mounting pads 54 can be disposed such that the datum are spaced from each other substantially equidistantly about the acetabular rim in one example. It is recognized that the datum can be spaced from each other substantially equidistantly when the mounting pads 54 are spaced from each other substantially equidistantly. It is also recognized that the datum can be spaced from each other substantially equidistantly when the mounting pads 54 are spaced from each other at variable distances. For instance, the relative position of the datum on each mounting pad can be different. The arms 56 can further extend from the guide member 52 such that the distal ends of the arms 56 are spaced from each other substantially equidistantly so as to be configured to couple to the equidistantly spaced datum. For instance, when the acetabular guide assembly 50 is viewed from the top plan view of FIG. 7A, the arms 56 can extend from guide member 52 in a configuration so as to define a number of respective angles, such as a first angle 80, a second angle 82, and a third angle s 84 defined by adjacent arms 56 of the plurality of the arms 56. For example, as illustrated in FIG. 4, a first arm 90 and a second arm 92 define the first angle 80 therebetween, the first arm 90 and a third arm 94 define the second angle 82 therebetween, and the second arm 92 and the third arm 94 define the third angle 84 therebetween. The angles 80, 82, and 84 can be substantially equal to each other.

It is recognized that the acetabular guide assembly 50 can include any number of mounting pads 54 and arms 56 as desired. Further, each mounting pad 54 can include a single datum. Therefore, the acetabular guide assembly 50 can include an equal number of arms 56 and mounting pads 54. Alternatively, one or more of the mounting pads 54 can include multiple datum. Therefore, the acetabular guide assembly 50 can include more arms 56 than mounting pads 54. Accordingly, while the acetabular guide assembly 50 is shown as including three mounting pads 54 and three arms 54, the number of arms and mounting pads can vary as desired.

Further, the mounting pads 54 can define equal mounting padprints along the underlying coxal bone 71. Alternatively, at least one or more of the mounting pads 54 can define a mounting padprint along the underlying coxal bone 71 that is different than that of at least one or more others of the mounting pads 54. For instance, at least one of the mounting pads 54 can be fitted onto a respective unique location of the coxal bone 71 that has both a healthy portion and an osteoporotic portion. The datum can overlie the osteoporotic portion, for instance when it is desirable for the datum to be equidistantly spaced from each other, and the at least one fastener 77 can be driven into the healthy portion.

Alternatively, referring now to FIG. 7B, the datum of the mounting pads 54 can be variably spaced about the acetabular rim, such that the circumferential distance between the mounting pads 54 of a first pair of adjacent mounting pads 54 is different than the circumferential distance between the mounting pads 54 of a second pair of adjacent mounting pads 54. Thus, the arms 56 can extend from the guide member 52 such that the distal ends of the arms 56 are similarly variably spaced from each other so as to be configured to couple to the equidistantly spaced mounting pads 54. In some examples, the magnitude of the third angle 84 can be greater than the magnitude of the second angle 82, which is greater than the magnitude of the first angle 80. Like many other dimensional characteristics described herein, the magnitude of the angles 80, 82, and 84 may be customized to any degree required for the particular patient. As described above, the arms 56 can be movable with respect to one or both of the guide member 54 and the respective mounting pads 54. Thus, the arms 56 can be moved to a position whereby the arms 56 can be coupled to the respective mounting pads 54 in the manner described above. For instance, when the mounting pads 54 define the bosses 65 described above, the arms 56 can be positioned and oriented such that the openings 69 are aligned with the bosses 65, and the arms 56 can then be coupled to the mounting pads 54.

In some embodiments, as illustrated in FIG. 8, the bottom surface 62 of the guide member 52 can define a relative position with respect to the top surface 68 of each mounting pad 54 along the longitudinal direction L. For instance, in one example, the bottom surface 62 can be offset a distance 76 from the top surface 68 of each mounting pad 54 along the longitudinal direction. That is, the bottom surface 62 may be non-coplanar with the top surface 68 of one or more of the mounting pads 54. As illustrated in FIG. 8, the distance 76 for each mounting pad 54 can be equal. In other examples, the distance 76 may be different such that the acetabular guide assembly 50 is positioned in the planned orientation and location, which has been predetermined to establish the desired inclination and anteversion planes of the acetabular orthopaedic prosthesis. Additionally, in still other example, the bottom surface 62 of the guide member 52 may be coplanar with the top surface 68 of each mounting pad 54. Further, in some examples, the guide member 52 may extend downwardly such that the bottom surface 62 of the guide member 52 is substantially equal to, higher than, or lower than the bottom surfaces 66 of the mounting pads 54. For example, the bottom surface 62 of the guide member 52 may be positioned medially relative to the mounting pads 54 when the acetabular reaming guide assembly 50 is coupled to the patient's coxal bone 71.

As further illustrated in FIG. 8, each of the mounting pads 54 has a height 74 along the longitudinal direction. In one example, the height 74 of each mounting pad 54 can be substantially equal to one another. In other embodiments, the height 74 of at least one of the mounting pads 54 can be different than the height 74 of at least one other one of the mounting pads 54 depending, for instance, on the contours of the unique locations of the underlying bone. The unique locations can be selected to ensure that the mounting pads and arms 56 define a stable construct for the guide member 52. In some examples, the length of the mounting pads 54 can be greater than the height of the mounting pads 54. Alternatively, the length of the mounting pads 54 can be less than the height of the mounting pads 54. In one example, the top surfaces 68 of the mounting pads 54 can be coplanar with each other. Alternatively, the top surfaces 68 of the mounting pads 54 can be offset with respect to each other along the longitudinal direction L.

As discussed above, the arms 56 are configured to couple the mounting pads 54 to the guide member 52. In the illustrative embodiment, the arms 56 are embodied as rectangular shafts, but may have other shapes and configurations in other embodiments. For example, the arms 56 may be straight, curved or bowed, angled, or the like in other embodiments. When viewed from the side elevation perspective of FIG. 8, an angle 78 can be defined between a bottom surface of each arm 56 and the bottom surface 62 of guide member 52. In the illustrative embodiment, each angle 78 is equal to one another. In other embodiments, each angle 78 may be different depending on the patient's anatomy and the desired angles of inclination and anteversion of the acetabular orthopaedic prosthesis. Additionally, when viewed from the top plan of FIGS. 7A-7B, each arm 56 extends a distance 86 from the guide member 52. It should be appreciated that in the illustrative embodiment of FIGS. 7A-7B, the arms 56 can extend the same distance 86 from the guide member 52. However, in other embodiments, the arms 56 may each extend a distance 86 that is different from one another depending on the patient's anatomy and the desired angles of inclination and anteversion of the acetabular orthopaedic prosthesis.

It should be appreciated that the acetabular guide assembly 50 can be adjustable by the orthopaedic surgeon to improve the coupling of the guide assembly 50 to the patient's underlying coxal bone 71. For example, when viewed from the side elevation perspective of FIG. 6B, each angle 78 defined between the bottom surface 62 of the guide member 52 and each arm 56 is adjustable in the manner described above so as to position the acetabular guide assembly 50 at the predetermined orientation and location. Additionally, an angle 124 is defined between each arm 56 and the sidewall 70 of each mounting pad 54. In the illustrative embodiment of FIG. 6B, the angle 124 is adjustable to position the acetabular guide assembly 50 at the desired predetermined location and orientation. In other embodiments, each angle 124 may or may not be adjustable depending on the patient's bony anatomy.

When viewed from the top plan of FIG. 7A, the angles 80, 82, and 84 defined between the arms 56 are also adjustable. The angles 80, 82, and 84 may be increased or decreased depending on the patient's bony anatomy to position the acetabular guide assembly 50 at the desired location and orientation. For example, any two of the arms 56 may be moved toward or away from each other. In other embodiments, the angles 80, 82, and 84 may or may not be adjustable depending on the patient's bony anatomy.

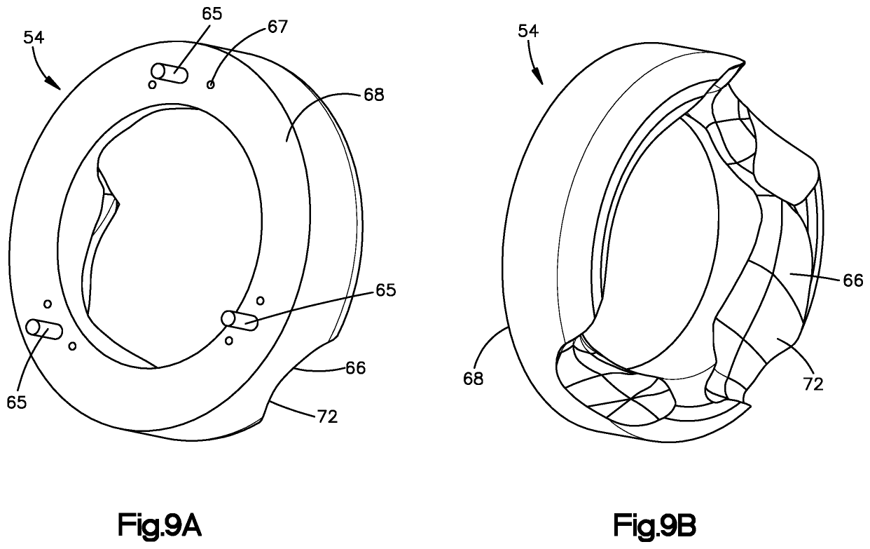

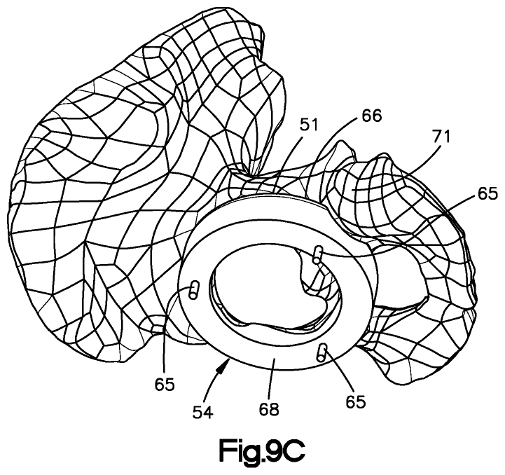

Referring now to FIGS. 9A-9C, it is recognized that the acetabular guide assembly 50 can include any number of mounting pads 54 and arms 56 as described above, including at least one. For instance, in one example, the acetabular guide assembly 50 can include a single monolithic mounting pad 54 that can surround at least a portion of the acetabular rim up to an entirety of the acetabular rim. In one example, the single mounting pad 54 can define an annulus. The reamer described herein can remove bone from the acetabulum disposed within the annulus with respect to a top plan view. For instance, the single mounting pad 54 can overlie a portion of the acetabular rim up to an entirety of the acetabular rim. Alternatively or additionally, the single mounting pad 54 can overlie a portion of the coxal bone 71 that is disposed proximate to the acetabular rim. The single mounting pad 54 can include a plurality of datum that are circumferentially spaced from each other as described above. That is, the single mounting pad 54 can include a plurality of attachment members each configured to couple to a respective different one of the arms 56 at a datum that is at least partially defined by the respective attachment member. For instance, the single mounting pad 54 can include a plurality of bosses 65 that, in turn, define the datum. Thus, the acetabular guide assembly 50 can be said to include at least one mounting pad 54 that defines a plurality of datum. The at least one mounting pad 54 can include a plurality (two or more) mounting pads 54, or can include the single mounting pad 54 illustrated in FIGS. 9A-9C.

The datum of the single mounting pad 54 can be equidistantly spaced about the mounting pad 54 or can be variably spaced from each other as described above. Further, the single mounting pad 54 can include any number of fixation apertures 67 as desired, including at least one that are predetermined at step 26 of FIG. 1 to be aligned with healthy bone. Accordingly, when a bone fastener is inserted through the fixation aperture 67, the bone fastener can be driven into healthy bone as preplanned at step 26. Further, an entirety of the bottom surface 66 of the single mounting pad 54 that faces the underlying coxal bone 71 can define a contour 72 that is configured to fit onto the underlying bone. Alternatively, the bottom surface 66 can include at least one patient specific contour 72 that is sized and dimensioned to fit onto the underlying bone, and at least one region that is designed to be spaced from the underlying bone. Thus, the bottom surface 66 can bear against at least a first predetermined region of the coxal bone 71 and can avoid contact with at least a second predetermined region of the coxal bone 71. For instance, the second region can be determined to be osteoporotic bone or can have some other relevant characteristic that indicates to the surgeon that the second region should avoid contact with the mounting pad 54.

It is recognized that the mounting pads 54 can advantageously be fabricated at step 32 of FIG. 1 using any suitable additive manufacturing process from any suitable material described herein. In one example, the mounting pads 54 can be fabricated using selective laser melting (SLM) techniques. Alternatively, the mounting pads 54 can be 3D printed. For instance, the mounting pads 54 can be fabricated using electron beam melting (EBM) techniques. Alternatively, the mounting pads 54 can be fabricated using layer deposition. In still other embodiments, the mounting pads 54 can be fabricated using rapid manufacturing. With each of these production techniques, the mounting pads 54 can be created without the further need for external fixation or attachment elements to keep the various structure of the mounting pads 54 together. Thus, the mounting pads 54 can each define a single monolithic component that is seamless. For instance, the mounting pads 54 can be devoid of connection seams at the interface between the outer surface of the mounting pad 54 and the boss 65, whereas mounting pads manufactured in accordance with conventional manufacturing techniques could include seams. For instance, the boss can be separately manufactured and screwed into or otherwise attached to the outer surface of the mounting pad 54. The lack of connection seams can render the mounting pads 54 produced using additive manufacturing techniques stronger than mounting pads 54 having connection seams.

Accordingly, the mounting pads 54 can be fabricated on-demand at step 30 of FIG. 1 with minimal delay once the model has been verified at step 28. Further, the additive material can rapidly create the unique positive contours of the bottom surface 66 using additive manufacturing. In a conventional manufacturing technique, a milling operation or other material removal operation can be complex and time consuming to create the uniquely contoured bottom surface. Further still, the fixation aperture 67 can be created in a single step without the use of sacrificial structure. For instance, certain conventional manufacturing techniques would overmold the mounting pad onto structures that are subsequently removed to reveal the fixation apertures. It should be appreciated, of course, that in alternative examples, the additive manufacturing process can, if desired, fabricate the mounting pads about sacrificial pins that can subsequently be removed so as to define the fixation apertures 67.

In one example, the arms 56, mounting pads 54, and guide member 52 can be each formed from separate pieces. For example, the arms 56 may be secured to the guide member 52 via suitable fasteners such as screws, bolts, adhesive, or the like. Alternatively, the arms 56 can be monolithic with the guide member 52. Alternatively, the guide member 52 and arms 56 can be formed as a monolithic component. Further, the mounting pads 54 and arms 56 can be formed as a monolithic component. Thus, the guide member 52, mounting pads 54, and arms 56 of the acetabular guide assembly 50 can all be formed as a single monolithic component. The guide member 52, mounting pads 54, and arms 56 can be formed from any suitable material such as a resilient plastic or metallic material. In one particular embodiment, the acetabular guide assembly 50 can be made from an implant-grade metallic material such as titanium or cobalt chromium.

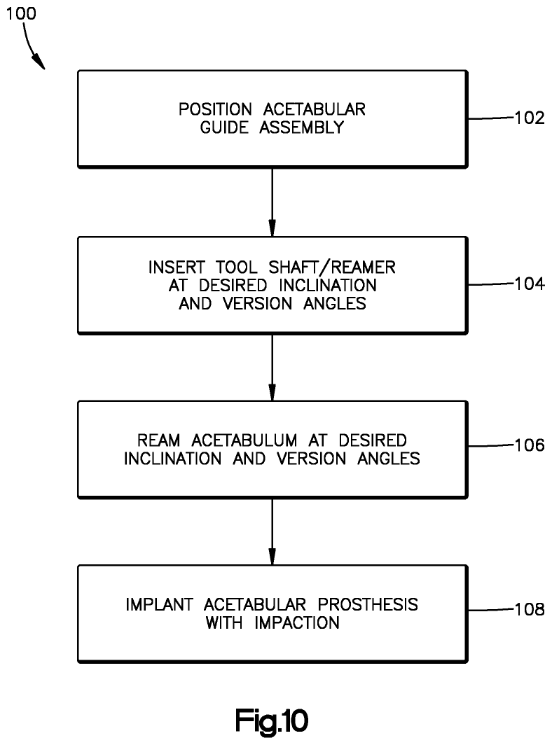

Referring to FIGS. 2 and 10, an orthopaedic surgical procedure 100 using the acetabular guide assembly 50 is illustrated. The surgeon may perform the procedure 100 in process step 32 of method 10, which is illustrated in FIG. 1 and described above. In process step 102, the surgeon positions the acetabular guide assembly 50 on the patient's coxal bone 71. Because the acetabular guide assembly 50 is customized to the particular patient, the guide assembly 50 is coupled to the patient's coxal bone 71 in a unique, predetermined location and orientation. For example, the respective bottom surface 66 of the at least one mounting pad 54 can be positioned such that such the at least one positive contour of the at least one mounting pad 54 is received in a respective at least one negative contour of the surface of the underlying coxal bone 71. Next, the arms 56 can be coupled to the at least one mounting pad 54. The arms 56 can then be coupled to the guide member 52. Alternatively, the arms 56 can be coupled to the guide member 52 prior to coupling the arms 56 to the at least one mounting pad 54.

Further, the arms 56 can extend from the guide member 52 and be coupled to the at least one mounting pad 54 prior to fitting the at least one mounting pad 54 onto the underlying coxal bone 71. In some examples, each mounting pad 54 may be uniquely keyed to a corresponding arm 56 such that each mounting pad is configured to be coupled to only one of the arms and no others of the arms. For instance, as illustrated in FIG. 5B, the coupling member of each mounting pad 54 can include a keyed surface 91, and the coupling member of each arm 56 can include a complementary keyed surface 89. The keyed surface 91 of each of the mounting pads 54 is configured to mate with the keyed surface 89 of only the respective arm 56 of the plurality of arms 56 so as to couple each of the mounting pads 54 with the respective arms 56. The keyed surface 91 of each of the mounting pads 54 are configured to interfere with the keyed surfaces 89 of all other arms 89 so as to prevent each mounting pad 54 from coupling to all arms 56 other than the respective arm 56. Similarly, the keyed surface 89 of each of the arms 56 is configured to mate with the keyed surface 91 of only one respective mounting pad 54. The keyed surface 89 of each of the arms 56 is configured to interfere with the keyed surfaces 91 of all other mounting pads 54 so as to prevent each arm 56 from coupling to any mounting pad 54 other than the respective mounting pad 54. As described above, the coupling member of the mounting pads 54 can be configured as a boss 65, and the coupling member of the arms 56 can be configured as an opening 69 configured to receive the boss 65. Thus, the boss 65 can define the keyed surface 91 of the mounting pad 54, and an inner surface of each of the arms 56 that define the openings 69 can define the boss 89 of the arms 56. The acetabular guide assembly 50 can include any suitable mechanical fasteners as desired, such as screws, to secure the mounting pads 54 to the arms 56 as desired.

Further, the keyed surfaces 89 and 91 can be configured such that the mounting pads 54 are coupled to the respective arms 56 in respective predetermined orientations such that the customized patient-specific contour 72 of each of the mounting pads 54 mates with the contour of the predetermined underlying portion of the corresponding the patient's coxal bone 71 (see FIGS. 9B-9C). The keyed surfaces 89 and 91 further prevent the mounting pads 54 from being coupled to the respective arms 56 in any other orientation than the predetermined orientation. Thus, when the arms 56 extend from the guide member 52 and are coupled to the respective mounting pads 54, the pre-assembled acetabular guide assembly 50 can be inserted into the patient, and the surgeon can locate the mounting pads 54 to their proper locations such that the contours 72 of the mounting pads mates with the predetermined underlying portion of the corresponding the patient's coxal bone 71. Accordingly, the mounting pads 54 are coupled to the respective arms 56 such that the contours 72 of the mounting pads 54 are disposed in respective predetermined locations and orientations that match the respective locations and contours of the underlying coxal bone 71. Thus, the surgeon can easily manipulate the preassembled acetabular guide assembly 50 until the respective contours 72 of the mounting pads 54 mate with the underlying contour of the coxal bone 71.

It is recognized that the openings 69 of the arms 56 be oriented along a direction such that the coupling structure of the arm 56 can be aligned to be coupled with the corresponding coupling structure of the mounting pads 54 without moving the arms 56 relative to the guide member 52. For instance, the arms 56 can be fixed relative to the guide member 52 in some examples. In other examples, the arms 56 can be movable with respect to each of the guide member 52 and the mounting pads 54 so as to align the respective coupling members of the arms 56 to the respective mounting pads 54.

When each mounting pad 54 is fitted over the underlying coxal bone 71 and the arms are coupled to the respective at least one mounting pad and to the guide member 52, the guide member is then supported by each mounting pad at a desired position and orientation with respect to the underlying coxal bone 71. After each mounting pad 54 has been fitted onto the underlying coxal bone 71, the fasteners 77 can be driven through the fixation apertures 67 and into the underlying coxal bone 71, thereby attaching the fixation apertures 67 to the underlying coxal bone 71. Additionally, in some embodiments, the surgeon may adjust the position of the acetabular guide assembly 50 pre-operatively or intraoperatively. For example, in those embodiments wherein each arm 56 is moveably secured to the guide member 52 and each mounting pad 54, the surgeon may adjust the position of the acetabular guide assembly 50 to adjust the orientation of the central axis 63 of the passageway 60. Once positioned, the acetabular guide assembly 50 defines the desired predetermined inclination and anteversion angles relative to the patient's acetabulum 51 intended for the acetabular orthopaedic prosthesis.

Referring now also to FIG. 11, in process step 104, the surgeon can insert a tool shaft 53 through the longitudinal passageway 60 defined by the guide member 52. In particular, the tool shaft 53 can be inserted through the longitudinal passageway 60 prior to fitting each mounting pad 54 onto the underlying coxal bone 71. Thus, it is not necessary to remove the guide member 52 from its position relative to the acetabulum 51 in order to attach the tool shaft 53 to the guide member 52. The tool shaft 53 can have an outer diameter that is substantially equal to the diameter of the longitudinal passageway 60. Accordingly, the tool shaft 53 is oriented along the central axis 66 of the longitudinal passageway 60. The tool shaft 53 can have a distal end that can be attached to a reamer 126. The tool shaft 53 can have a proximal end that can be driven to rotate about the central axis 66 of the longitudinal passageway 60 by a driving instrument. The tool shaft 53 is further translatable in the longitudinal passageway 60 to bring the reamer 126 against the underlying bone. The tool shaft 53 can be inserted through the passageway of the guide member 52 prior to causing the guide member 52 to be supported by each mounting pad 56. Thus, the reamer 126 can be disposed between the bottom surface 62 of the guide member 52 when the guide member 52 is supported by each mounting pad 56. As described above, each mounting pad 56 can be defined by a single mounting pad or a plurality of mounting pads 56.

The surgeon can then begin reaming the patient's acetabulum 51 by rotating the tool shaft 53 in process step 106. It should be appreciated that because the guide member 52 is supported by each one mounting pad at a predetermined location and orientation, the longitudinal passageway 60 is similarly disposed in a predetermined location and orientation based on the desired anteversion and inclination angle of the acetabular prosthesis. Thus, the reaming of the patient's acetabulum 51 is guided by the longitudinal passageway 60 so as to size the patient's acetabulum 51 to receive the acetabular prosthesis according to the desired predetermined anteversion and inclination angles.

The acetabular guide assembly 50 can further include a stop member 81 that is supported by the tool shaft 53. The stop member 81 can be configured to contact the guide body 58 as the tool shaft is driven distally toward the underlying acetabulum 51, thereby preventing further distal movement of the tool shaft 53 toward the underlying acetabulum 51. For instance, the stop member 81 can be configured to contact the top surface 61 of the guide body 58 to prevent further distal movement of the tool shaft 53 toward the underlying acetabulum 51. Thus, the reamer 126 can be driven into the acetabulum a predetermined distance until the stop member 81 contacts the guide body 58. It is recognized that it can be desired to ream different patients at different depths depending on the patient's anatomy. Thus, the stop member 81 can be disposed at an adjustable position along the length of the tool shaft 53. For instance, a set screw 83 can extend into the stop member 81 and can be tightened against the tool shaft 53 so as to positionally fix the stop member 81 with respect to the tool shaft 53. The set screw 83 can be loosened to allow for the stop member 81 to translate along the tool shaft 53 to a desired stop location. The set screw 83 can be tightened against the tool shaft 53 to fix the stop member 81 at the desired stop location. The tool shaft 53 can include a plurality of markings 79 that can indicate spatial relationships with respect to the distal end of the tool shaft 53. Accordingly, when the reamer 126 is attached to the distal end of the tool shaft 53, the markings can indicate a distance to the reamer 126. Therefore, during operation, the stop member 81 can be adjusted to a position along the tool shaft 53 to determine the depth that the reamer 126 will ream into the underlying acetabulum 51.

As illustrated in FIGS. 10 and 12, after the surgeon has reamed the patient's bone using the cannulated reamer 126, the surgeon may position an acetabular prosthesis 140 to the reamed acetabulum 51 of the patient in process step 108. The acetabular prostheses 140 can include an acetabular cup 142 and a bearing liner 144 received within the acetabular cup 142. The acetabular prosthesis 140 may be implanted via use of an impactor 148. In the illustrative embodiment, the impactor 148 has a substantially spherical-shaped impaction surface, but can have any suitable alternatively shaped impaction surface as desired. The impactor 148 is includes a centrally-positioned passageway 149, which is sized to receive the tool shaft 53. During operation, the reamer 126 can be removed from the distal end of the tool shaft 53, and the impactor 148 can be attached to the tool shaft 53. For instance, the distal end of the tool shaft 53 can be inserted through the passageway of the tool shaft 53. The tool shaft 53 can then be attached to the acetabular prosthesis 140. For instance, in one embodiment, a threaded distal end of the tool shaft 53 is threadedly mated in a threaded aperture of the acetabular prosthesis. The threaded aperture of the acetabular prosthesis 140 can be centrally located. It is appreciated, however, that any suitable attachment mechanism can attach the tool shaft 53 to the acetabular prosthesis 140. Thus, the impactor 148 is aligned with the rim of the acetabular prosthesis 140 along the longitudinal direction L. Accordingly, the guide member 52 can guide the impactor 148 to translate along the tool shaft 53 and contact the acetabular prosthesis 140, thereby driving the acetabular prosthesis to a seated position in the underlying acetabulum.

As described above with respect to FIG. 11, the stop member 81 can be configured to contact the guide body 58 when the tool shaft 53 is attached to the acetabular prosthesis 140, and the acetabular prosthesis is fully seated in the underlying acetabulum 51. This prevents further distal movement of the tool shaft 53, and thus of the impactor 148, toward the underlying acetabulum 51 if the impactor 148 is further driven against the acetabular prosthesis 140 after the acetabular prosthesis 140 has been fully seated. The stop member 81 can be disposed at an adjustable position along the length of the tool shaft 53. The set screw 83 can be loosened to allow for the stop member 81 to translate along the tool shaft 53 to a desired stop location. When the acetabular prosthesis 140 is attached to the distal end of the tool shaft 53, the markings can indicate a depth along which the acetabular prosthesis 140 can be driven against the underlying acetabulum 51 by the impactor 148. Therefore, during operation, the stop member 81 can be adjusted to a position along the tool shaft 53 to determine the depth that the impactor 148 is able to drive the acetabular prosthesis 140 into the underlying acetabulum 51. It should be appreciated that because the acetabular prosthesis 140 is implanted using the longitudinal passageway 60 as a guide, the acetabular prosthesis 140 is implanted at the predetermined location and orientation (e.g., at the predetermined inclination and anteversion angles).

The surgeon can impact the impactor 148 (e.g., via use of a surgical hammer) to cause the acetabular prosthesis 140 to seat into the patient's surgically-prepared acetabulum 51. Of course, in other embodiments, other devices and tools may be used to implant the acetabular prosthesis 140 as will be described in more detail below. Once the acetabular prosthesis 140 is implanted, the acetabular guide assembly 50 can be removed from the coxal bone 71. In particular, tool shaft 53 is detached from the acetabular prosthesis 140. Further, the fasteners 77 are removed from the coxal bone 71 and the at least one mounting pad 54. Next, the at least one mounting pad 54 is removed from the coxal bone 71. If desired, the guide member 52 can be removed from the arms 56 prior to removing the at least one mounting pad 54 from the coxal bone 71. Alternatively or additionally, the arms 56 can be removed from the at least one mounting pad 54 prior to removing the at least one mounting pad 54 from the coxal bone 71.

Referring now to FIG. 13, it is recognized that any suitable alternative procedure can be performed to implant the acetabular prostheses 140 at the predetermined location and orientation (e.g., at the predetermined inclination and anteversion angles). For instance, an alternative orthopaedic surgical procedure 200 using the acetabular guide assembly 50 is illustrated. The surgeon may perform the procedure 200 in process step 32 of method 10, which is illustrated in FIG. 1 and described above. In process step 202, the surgeon positions the acetabular guide assembly 50 on the patient's coxal bone 71. Because the acetabular guide assembly 50 is customized to the particular patient, the guide assembly 50 is coupled to the patient's coxal bone 71 in a unique, predetermined location and orientation. For example, the respective bottom surface 66 of the at least one mounting pad 54 can be positioned such that such the at least one positive contour of the at least one mounting pad 54 is received in a respective at least one negative contour of the surface of the underlying coxal bone 71. Next, the arms 56 can be coupled to the at least one mounting pad 54. The arms 56 can then be coupled to the guide member 52. Alternatively, the arms 56 can be coupled to the guide member 52 prior to coupling the arms 56 to the at least one mounting pad 54. Further, the arms 56 can be coupled to the at least one mounting pad 54 prior to fitting the at least one mounting pad 54 onto the underlying coxal bone 71.

It is recognized that the openings 69 of the arms 56 be oriented along a direction such that the coupling structure of the arm 56 can be aligned to be coupled with the corresponding coupling structure of the at least one mounting pad 54 without moving the arms 56 relative to the guide member 52. For instance, the arms 56 can be fixed relative to the guide member 52 in some examples. In other examples, the arms 56 can be movable with respect to each of the guide member 52 and the at least one mounting pad 54 so as to align the respective coupling members of the arms 56 to the respective at least one mounting pad 54.

When the at least one mounting pad 54 is fitted over the underlying coxal bone 71 and the arms are coupled to the at least one mounting pad and to the guide member 52, the guide member is then supported by the at least one mounting pad at a desired position and orientation with respect to the underlying coxal bone 71. After the at least one mounting pad 54 has been fitted onto the underlying coxal bone 71, the fasteners 77 can be driven through the fixation apertures 67 and into the underlying coxal bone 71, thereby attaching the fixation apertures 67 to the underlying coxal bone 71. Additionally, in some embodiments, the surgeon may adjust the position of the acetabular guide assembly 50 pre-operatively or interoperatively. For example, in those embodiments wherein each arm 56 is moveably secured to the guide member 52 and each mounting pad 54, the surgeon may adjust the position of the acetabular guide assembly 50 to adjust the orientation of the central axis 63 of the passageway 60. Once positioned, the acetabular guide assembly 50 defines the desired predetermined inclination and anteversion angles relative to the patient's acetabulum 51 intended for the acetabular orthopaedic prosthesis.

In process step 204, the surgeon inserts a drill bit of an orthopaedic drill through the passageway 60 of the guide member 52 of the acetabular guide assembly 50. The surgeon drills a pilot hole in the patient's acetabulum 51 using the guide member 52. It should be appreciated that the pilot hole is oriented to position the acetabular orthopaedic prosthesis at the desired inclination and anteversion angles. Thereafter, the surgeon may remove the drill bit from the passageway 60.

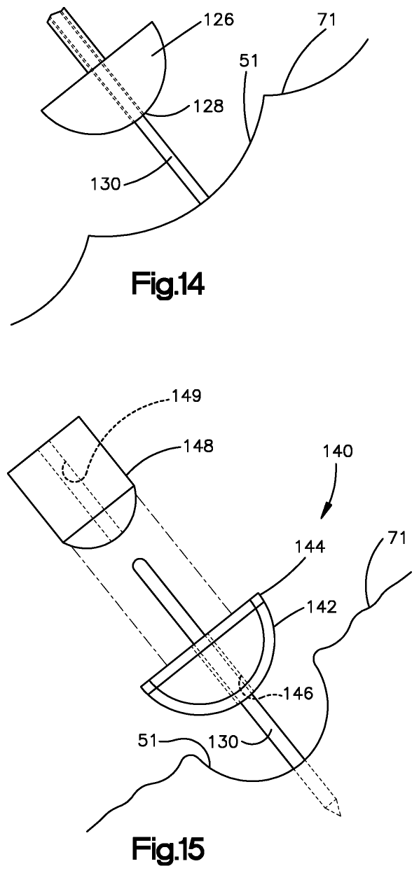

In process step 206, the surgeon inserts a guide pin 130 (see FIG. 14) through the passageway 60 of the guide member 52 and into the pilot hole defined in the patient's acetabulum 51. In this regard, the tool shaft described above can be configured as the guide pin 130. The guide pin is then screwed or otherwise secured in the patient's acetabulum 51. After securing the guide pin to the patient's acetabulum 51, the surgeon removes the acetabular guide assembly 50, leaving the guide pin secured to the patient's acetabulum 51. Alternatively, in some embodiments, the surgeon may remove the acetabular guide assembly 50 after establishing the pilot hole in the patient's acetabulum 51. The surgeon may subsequently secure the guide pin in the pilot hole without the use of the guide member 52.

In process step 208, the surgeon advances a cannulated reamer (see FIG. 14) over the guide pin 130. As shown in FIG. 14, the cannulated reamer 126 includes a centrally-defined cannula or passageway 128 sized to receive a guide pin 130. The surgeon may advance the cannulated reamer 126 over the guide pin 130 to begin reaming the patient's acetabulum 51. It should be appreciated that because the guide pin 130 was secured to the patient's acetabulum 51 in a predetermined location and orientation based on the desired anteversion and inclination angle of the acetabular prosthesis, the reaming of the patient's acetabulum 51 is guided so as to size the patient's acetabulum 51 to receive the acetabular prosthesis according to the desired anteversion and inclination angles.

In some embodiments, the guide pin 130 may also be used as a guide during the implantation of an acetabular prosthesis. That is, as illustrated in FIG. 15, after the surgeon has reamed the patient's bone using the cannulated reamer 126, the surgeon may position an acetabular prosthesis 140, which may include an acetabular cup 142 and a bearing liner 144 received within the acetabular cup 142, over the guide pin 130. The acetabular prosthesis 140 includes an aperture 146, which may be threaded or non-threaded, positioned at the dwell point of the acetabular cup 142. The acetabular prosthesis 140 is positioned such that the guide pin 130 is received through the aperture 146. The acetabular prosthesis 140 may subsequently be slid down the guide pin 130 to the surgically-prepared acetabulum 51 of the patient.

The acetabular prosthesis 140 may be implanted via use of an impactor or inserter 148. In the illustrative embodiment, the impactor 148 is substantially cylindrical in shape and has an outer diameter substantially equal to the outer diameter of the acetabular prosthesis 140. The impactor 148 is includes a centrally-positioned passageway 149, which is sized to receive the end of the guide pin 130 such that the impactor 148 may be positioned over the acetabular prosthesis 140. When so positioned, the impactor 148 contacts the rim of the acetabular prosthesis 140. The surgeon may then impact the impactor 148 (e.g., via use of a surgical hammer) to cause the acetabular prosthesis 140 to seed into the patient's surgically-prepared acetabulum 51. Of course, in other embodiments, other devices and tools may be used to implant the acetabular prosthesis 140 using the guide pin 130 as a guide. For example, in some embodiment, the impactor may be embodied as, or otherwise include, a stem configured to be received in the aperture 146. In such embodiments, the stem and aperture 146 are threaded. In addition, the stem is cannulated and configured to receive the guide pin 130 therein. In should be appreciated that in such embodiments, the aperture 146 has a greater diameter than the guide pin 130 to allow the stem of the impactor to be received therein. Regardless, once the acetabular prosthesis 140 is implanted, the guide pin 130 may be removed. It should be appreciated that because the acetabular prosthesis 140 is implanted using the guide pin 130 as a guide, the acetabular prosthesis 140 is implanted at the predetermined location and orientation (e.g., at the predetermined inclination and anteversion angles).

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An acetabular guide assembly comprising:
a generic guide member that includes a guide body and a passageway extending through the guide body along a central axis;
at least one additively manufactured mounting pad defining a top surface and a bottom surface opposite the top surface, wherein the bottom surface has a patient-specific positive contour that matches a negative contour surface of a coxal bone proximate to an acetabulum;
a plurality of arms that are configured to extend from the guide body to the at least one mounting pad, so as to support the guide member relative to the at least one mounting pad at a predetermined position and orientation, wherein the central axis has a predetermined relationship with respect to planes of anteversion and inclination,
wherein the at least one mounting pad includes a plurality of coupling members that are each configured to couple to at least one of the plurality of arms, and an entirety of the mounting pad is seamless.

2. The acetabular guide assembly of claim 1, wherein the at least one mounting pad comprises a plurality of mounting pads whose bottom surface, respectively, is contoured so to fit onto a unique portion of the coxal bone.

3. The acetabular guide assembly of claim 2, wherein the coupling members have a predetermined spatial relationship with each other such that the central axis of the guide member supported by the mounting pads has the predetermined relationship with respect to the planes of anteversion and inclination.

4. The acetabular guide assembly of claim 3, wherein the upper surfaces of the mounting pads are substantially coplanar with each other when coupled to the arms, respectively, that in turn are coupled to the guide member.

5. The acetabular guide assembly of claim 2, wherein the mounting pads and arms define respective keyed surfaces, such that each of the mounting pads is configured to be coupled to a respective one of the arms and no other arm, wherein the keyed surfaces allow each of mounting pads to couple to the respective one of the arms in a predetermined orientation, and prevent each of the mounting pads from coupling to the respective one of the arms in any orientation other than the predetermined orientation.

6. The acetabular guide assembly of claim 1, wherein the at least one mounting pad comprises a single monolithic mounting pad that includes the plurality of coupling members and at least one patient-specific contour at its bottom surface.

7. The acetabular guide assembly of claim 1, wherein the at least one additively manufactured mounting pad comprises a plurality of mounting pads, and each of the mounting pads is uniquely keyed to a corresponding one of the arms so as to be located at a first predetermined location and oriented in a first predetermined orientation.

8. The acetabular guide assembly of claim 1, further comprising a tool shaft that is sized to be received in the passageway, and configured to rotate and translate in the passageway.

9. An acetabular implantation system comprising:
the acetabular guide assembly of claim 8; and
at least one of a reamer and an impactor that are configured to selectively couple to the tool shaft.

10. The acetabular implantation system of claim 9, wherein the tool shaft further comprises a stop member that is configured to abut the guide member so as to limit translation of the tool shaft in the passageway.

11. First and second acetabular guide assemblies, each comprising:
a guide body that defines a longitudinal passageway, wherein the guide body of the first acetabular guide assembly is substantially identical to the guide body of the second acetabular guide assembly;
a plurality of additively manufactured mounting pads each having respective patient-specific positive contours that match corresponding negative contoured surfaces at unique locations of a coxal bone proximate to an acetabulum, wherein the patient-specific positive contours of the mounting pads of the first acetabular guide assembly are all different than the patient-specific positive contours of the mounting pads of the second acetabular guide assembly; and
a plurality of arms configured to extend from the guide body to the plurality of mounting pads, wherein the arms of the first acetabular guide assembly are configured to support the guide body of the first acetabular guide assembly at a first predetermined location and orientation with respect to the acetabulum of a first patient, and the arms of the second acetabular guide assembly are configured to support the guide body of the second acetabular guide assembly at a second predetermined location and orientation with respect to the acetabulum of a second patient.

12. The first and second acetabular guide assemblies of claim 11, wherein each of the mounting pads of the first acetabular guide assembly is uniquely keyed to a corresponding one of the arms of the first acetabular guide assembly so as to be located at a first predetermined location and oriented in a first predetermined orientation, and
wherein each of the mounting pads of the second acetabular guide assembly is uniquely keyed to a corresponding one of the arms of the second acetabular guide assembly so as to be located at a second predetermined location and oriented in a second predetermined orientation.

13. The first and second acetabular guide assemblies of claim 12, each further comprising a tool shaft configured to rotate and translate in a passageway of the guide body, the tool shaft further configured to selectively couple to a reamer and an impactor.

14. The first and second acetabular guide assemblies of claim 13, wherein the tool shaft comprises a stop member configured to abut the guide body so as to limit translation of the tool shaft in the passageway.

15. A method for preparing an acetabulum for an implantation of an acetabular prosthesis, the method comprising:
fitting a bottom surface of at least one additively manufactured mounting pad onto a preplanned portion of a coxal bone proximate to the acetabulum, such that a guide member is supported relative to the at least one mounting pad at a predetermined location and orientation with respective to planes of anteversion and inclination; and
inserting a tool shaft through the passageway, and
rotating the tool shaft about the central axis so as to ream the acetabulum with a reamer attached to the tool shaft.

16. The method of claim 15, further comprising the step of guiding the tool shaft to rotate about the central axis during the rotating step.

17. The method of claim 15, further comprising the steps of:
removing the reamer from the tool shaft; and
attaching an impactor to the tool shaft, wherein the impactor is translatable along the central axis so as to drive the acetabular prostheses into the acetabulum.

18. The method of claim 15, wherein the fitting step comprises fitting multiple contours onto respective unique predetermined locations of the coxal bone.

19. The method of claim 18, wherein the at least one additively manufactured mounting pad comprises a plurality of additively manufactured mounting pads, the method further comprising the step of coupling each of the plurality of additively manufactured mounting pads to the respective one of a plurality of arms in a predetermined orientation while preventing each of the plurality of additively manufactured mounting pads from being coupled to the respective one of the plurality of arms in any orientation other than the predetermined orientation.

20. The method of claim 19, wherein the step of coupling each of the plurality of additively manufactured mounting pads to the respective one of the plurality of arms is performed prior to the fitting step.
